(19) United States
(12) Patent Application Publication
(10) Pub. No.: US 2018/0185431 A1
(43) Pub. Date: Jul. 5, 2018

(54) HOVENIAE SEMEN CUM FRUCTRUS EXTRACT COMPOSITIONS AND METHOD OF USE

(71) Applicant: ARIBIO Co., Ltd., Sungnam (KR)

(72) Inventors: Ilje CHO, Daegu (KR); Joowan KIM, Changwon-si (KR); Jaijun JUNG, Sungnam-si (KR); Soohyun SUNG, Seoul (KR); Saekwang KU, Daegu (KR)

(73) Assignee: ARIBIO Co., Ltd., Sungnam (KR)

(21) Appl. No.: 15/850,439

(22) Filed: Dec. 21, 2017

Related U.S. Application Data
(60) Provisional application No. 62/440,094, filed on Dec. 29, 2016.

(51) Int. Cl. A61K 36/72; A61P 1/16; A23L 33/105; A23L 33/00; B01D 11/02

(52) U.S. Cl. CPC A61K 36/72; A61P 1/16; A23L 33/105; A23L 33/30; A23V 2002/00; A61K 2236/15; A61K 2236/331; A61K 2236/53; A61K 2236/51; B01D 11/0288

(57) ABSTRACT

The invention relates to compositions having Hoveniae semen cum fructus extract, and methods of using such compositions to protect against oxidation or oxidative stress. The invention also relates to the use of compositions having Hoveniae semen cum fructus extract to protect the liver against liver disease induced by alcohol, chemical, or stress, such as oxidation or oxidative stress, and other toxic conditions, or mixture thereof.

### CROSS-REFERENCE TO RELATED APPLICATIONS

This application claims the benefit of U.S. provisional patent application Ser. No. 62/440,094, filed Dec. 29, 2016, the contents of which is incorporated herein by reference.

### FIELD OF THE INVENTION

The invention relates to compositions comprising Hoveniae semen cum fructus extract, and methods of using such compositions to protect against oxidation or oxidative stress.

The invention also relates to the use of compositions comprising Hoveniae semen cum fructus extract to protect the liver against alcohol, drug, stress, and other chemicals induced liver disease, oxidation or oxidative stress, and other toxic conditions.

### BACKGROUND

The liver is an important organ actively involved in metabolic functions and is a frequent target of a number of toxicants. It is well known that a substantial increase in steatosis and fibrosis usually leads to potentially lethal cirrhosis of the liver in humans.

Alcohol-induced liver disease, which ranges from simple fatty liver to cirrhosis and hepatocellular carcinoma, remains a major cause of liver-associated mortality worldwide. Long-term alcohol use potentially results in serious illnesses, including alcoholic fatty liver, hypertriglyceridaemia, cirrhosis, cardiovascular disease and inflammation of the pancreas [Ponnappa and Rubin, 2000].

It is also known that ethanol (EtOH) administration causes accumulation of reactive oxygen species (ROS), including superoxide, hydroxyl radical, and hydrogen peroxide [Nordmann, 1994]. ROS, in turn, cause lipid peroxidation of cellular membranes, and protein and DNA oxidation, which results in hepatocyte injury [Kurose et al., 1997; Rouach et al., 1997]. The potential harmful effects of these species are controlled by the cellular antioxidant defense system [Bondy and Orozco, 1994]. Reduced glutathione (GSH) is the predominant defense against ROS/free radicals in different tissues of the body [DeLeve and Kaplowitz, 1991]. In addition, antioxidant enzymes, such as superoxide dismutase (SOD), catalase (CAT), glutamate cystein ligase (GCL) and Hemeoxygenase-1 (HO1) are essential in both scavenging ROS/free radicals and maintaining cellular stability [Somani, 1996]. Under normal conditions, reductive and oxidative capacities of the cell (redox state) favor oxidation. However, when the generation of ROS in cells impairs antioxidant defenses or exceeds the ability of the antioxidant defense system to eliminate them, oxidative stress results [Jenkins and Goldfarb, 1993].

Oxidative stress and lipid peroxidation are predominantly generated through the induction of cytochrome P450 (CYP) 2E1 [Wang et al., 2013]. A key role for this enzyme in ethanol-induced liver injury has been demonstrated by its inhibition through chlormethiazole and by the finding that CYP2E1 knock-out mice do not show evidence of ethanol-induced liver disease [Lu et al., 2008].

It is further well established that increased ROS and electrophiles induce a series of antioxidant genes via activation of antioxidant response elements (AREs). ARE-driven gene expression is mainly regulated by NF-E2-related factor-2 (Nrf2), which are essential transcription factors that regulate the expression of major antioxidant enzymes including glutathione S-transferase A1/2, hemeoxygenase 1, UDP-glucuronosyl transferase 1A, NAD(P)H dehydrogenase quinone 1 (NQO1), and γ-glutamylcysteine synthetase [Kobayashi and Yamamoto, 2005].

Another major consequence of EtOH metabolism is lipid accumulation in the liver. EtOH metabolism changes the NAD/NADH ratio, which has important consequences on fuel utilization in the liver, favoring the synthesis of fatty acids and inhibiting their oxidation [Nagy, 2004; Zeng and Xie, 2009]. Sterol regulatory element-binding protein-1c (SREBP-1c) and peroxisome proliferator-activated receptor (PPAR) α, two nuclear transcription regulators controlling lipid metabolism, are involved in the development of alcoholic fatty liver [Yang et al., 2013; Wang et al., 2013]. EtOH administration activates hepatic SREBP-1c gene and its target genes: fatty acid synthase (FAS), stearoyl-CoA desaturase 1 (SCD1), and acetyl-CoA carboxylase 1 (ACC1), which promotes de novo fatty-acid synthesis [Yang et al., 2013; Wang et al., 2013]. It also increases the expression of genes for PPARγ and diacylglycerol acyltransferase (DGAT) 2, which promotes triglyceride (TG) synthesis [Yu et al., 2003; Herziget al., 2003; Wada et al., 2008; Yang et al., 2013; Wang et al., 2013]. EtOH decreases the expression of mRNA encoding PPARγ, acyl-CoA oxidase (ACO) and carnitine palmitoyltransferasel (CPT1), which leads to the inhibition of fatty acid oxidation [Reddy and Mannaerts, 1994; Yang et al., 2013; Wang et al., 2013].

EtOH-mediated experimental liver damaged rodents have been used for detecting the hepatoprotective effects of various herbal extracts or their chemical components based on the changes of body and liver weights. Histopathology of the liver with blood chemistry like serum aspartate aminotransferase (AST), alanine aminotransferase (ALT), alkaline phosphatase (ALP), TG, γ-glutamyl transferase (γ-GTP), and albumin with hepatic TG contents, hepatic lipid peroxidative makers, mRNA expression of hepatic lipogenic genes or genes involved in fatty acid oxidation, and especially histopathological changes of hepatic parenchyma have been used as critical end points of EtOH-mediated hepatic damages in rodent models [Jafri et al., 1999; Kumar et al., 2002; Saravanan et al., 2006; Song et al., 2006; Devipariya et al., 2007; Kaviarasan and Anuradha, 2007; Yang et al., 2013; Wang et al., 2013].

Early research on the pathogenesis of the alcohol-induced liver disease primarily focused on alcohol metabolism-related oxidative stress, malnutrition, and activation of Kupffer cells by endotoxin. Despite improved understanding of the pathophysiology of alcohol-induced liver disease, there is no Food and Drug Administration-approved drug for the specific treatment of alcohol-induced liver disease. Therefore the development of effective therapeutic strategies for alcohol-induced liver disease is pivotal. Among the several pathways involved in the pathogenesis of alcohol-induced liver disease, one of the central pathways is through the induction of CYP2E1 by alcohol, leading to the induction oxidative stress including lipid peroxidation, mitochondrial dysfunction and so on.

As such, there is a need for agents which enhance antioxidant capacities for the treatment of alcohol-induced liver disease.

### SUMMARY OF THE INVENTION

Hoveniae Semen Cum Fructus (“HSCF”) is the dried peduncle of Hovenia dulcis Thunb. (Rhamnaceae).

A new and novel composition for protecting the liver against oxidative stress caused by alcohol or other toxic conditions is provided comprising Hoveniae semen cum fructus extract.

A new and novel method for protecting liver from damage such as oxidative stress caused by alcohol or other toxic condition such as chemical, stress, or combination thereof, is also provided comprising the step of administering to a patient a composition comprising Hoveniae semen cum fructus extract.

In an in vitro study, the cytoprotective effect of HSCF extracts on oxidative stress-mediated cell damage was evaluated by using HepG2 cells. Cytotoxic effect of HSCF extracts was observed in HepG2 cells and determined IC₅₀ (50% inhibitory concentration). Cytoprotective effect of sub-lethal dose of HSCF extracts was evaluated by using tert-butyl hydroperoxide (tBHP)-induced cellular damage model. Also, the present invention provides whether NF-E2-related factor-2 (Nrf2) was transactivated by HSCF extracts. antioxidant capacity of HSCF extracts was evaluated as superoxide dismutase (SOD) and catalase (CAT) activities, and expression of antioxidant genes—glutamate; cysteine ligase catalytic subunit (GCLC), hemeoxygenase-1 (HO1) and NAD(P)H dehydrogenase quinone 1 (NQO1). Up to 1,000 μg/ml concentration of HSCF extracts did not show any cytotoxic effect in HepG2 cells. 300 and 1,000 μg/ml of HSCF extracts significantly protected HepG2 cells from oxidative stress-mediated cell death by tBHP. As a molecular mechanism of HSCF extracts 1,000 μg/ml treatment significantly increased Nrf2 transactivation and induced its target genes expression (GCLC, HO1 and NQO1). Furthermore, 1000 μg/ml HSCF extracts enhanced SOD activity. Although 300 and 1,000 μg/ml HSCF extracts treatment tended to slightly increase catalase activity, those increases were not statistically significant. The results of this in vitro study support that HSCF extracts have favorable hepatoprotective effects against oxidative stress through Nrf2-mediated antioxidant gene induction.

Furthermore, an in vivo study found that oral administration of 500, 250, and 120 mg/kg of HSCF favorably protected against liver damages from subacute mouse EtOH intoxication. Hoveniae Semen Cum Fructus extract demonstrated potent anti-inflammatory and anti-steatosis properties through augmentation of the hepatic antioxidant defense system, mediated by Nrf2 activation and down-regulation of the mRNA expression of hepatic lipogenic genes or up-regulation of the mRNA expression of genes involved in fatty acid oxidation.

A certain embodiment of the present invention provides HSCF extract is a potent hepatoprotective agent for protecting against liver diseases, with less toxicity than known conventional liver medication.

### BRIEF DESCRIPTION OF THE DRAWINGS

FIG. 1 shows the effects of HSCF extracts on the HepG2 cell viabilities.

FIG. 2 shows the effects of HSCF extracts on tBHP-mediated HepG2 cell cytotoxicity.

FIG. 3 shows the effects of HSCF extracts on ARE-driven luciferase activity.

FIG. 4 shows the effects of HSCF extracts on the mRNA expression of antioxidant genes.

FIG. 5 shows the effects of HSCF extracts on CAT activity.

FIG. 6 shows the effects of HSCF extracts on SOD activity.

FIG. 7 lists the oligonucleotides primers used for RT-qPCR in the study.

FIG. 8 lists primary antisera and detection kits used in immunohistochemistry.

FIG. 9 displays the body weight in mice with subacute EtOH-induced intoxication in the study.

FIG. 10 displays the changes in the serum biochemistry in subacute EtOH-treated mice in the study.

FIG. 11 displays the hepatic TG and TNF-α contents with hepatic CYP450 2E1 activity in subacute EtOH-treated mice in the study.

FIG. 12 displays the hepatic lipid peroxidation and antioxidant defense systems in subacute EtOH-treated mice in the study.

FIG. 13 displays the RT-PCR analysis of hepatic lipogenic genes, genes involved in fatty acid oxidation or master transcription factor of antioxidant genes, Nrf2 in subacute EtOH-treated mice in the study.

FIG. 14 displays the hepatic tissue histopathological analysis in subacute EtOH-treated mice in the study.

FIG. 15 shows an exemplary histological images of the liver, taken from subacute EtOH-treated mice in the study.

FIG. 16 shows an exemplary histological images of NT and 4-NHE-immunoreactivities in the liver sections, taken from subacute EtOH-treated mice in the study.

### DETAILED DESCRIPTION OF THE INVENTION

Hoveniae semen cum fructus extract is derived from Hovenia dulcis Thunb , which is a tree native to the Himalayas, China, Korea and Japan that can grow to 30 ft in height, with broadly ovate, glossy dark-green leaves.

As will be discussed below, one embodiment of the present invention provides composition comprising a Hoveniae semen cum fructus extract.

Preparation of the Extract of Hoveniae Semen cum Fructus

The extract of Hoveniae semen cum fructus employed in the present invention is prepared by using extraction method known to the art in the field.

In an alternative embodiment, the extract of Hoveniae semen cum fructus employed in the present invention is prepared by the following steps:

A method for preparing an extract of Hoveniae semem cum fructus comprising:

grinding Hoveniae semem cum fructus to obtain ground Hoveniae semem cum fructus;

extracting the ground Hoveniae semem cum fructus obtained from step (i) with hot water one to four times at 40-100° C. for 2-10 hours; filtering the mixture to obtain a filtrate;

removing the water from the filtrate under reduced pressure to obtain a residue; and drying and standardizing the residue using a spray drier 7.4-14.2 ug/g quercetin to obtain an extract of Hoveniae semem cum fructus.

In another embodiment, a method for preparing an extract of Hoveniae semem cum fructus comprising:

grinding Hoveniae semem cum fructus to obtain ground Hoveniae semem cum fructus;

extracting the ground Hoveniae semem cum fructus obtained from step (i) with hot water twice at 95° C. for 4 hours;

filtering the mixture to obtain a filtrate;

removing the water from the filtrate under reduced pressure to obtain a residue; and

drying and standardizing the residue using a spray drier 11.84 ug/g quercetin to obtain an extract of Hoveniae semem cum fructus.

A certain embodiment of the present invention also provides a method for treating alcohol, drug, stress, or other chemical induced liver disease comprising the step of administering to a patient a composition comprising a Hoveniae semen cum fructus extract.

The composition of the present invention may be a pharmaceutical or a dietary supplement, and can be administered using any convention means (i.e., oral, injection, submuccal, parenteral, etc.).

The composition of the present invention can also include any known additives or excipients (i.e., binders, surfactants, etc.) conventionally used to form the desired administration form (i.e., tablet, capsule, liquid, powder, etc.)

The present invention was confirmed by the result of the in vitro and in vivo studies. The results of those separate studies are discussed in the sections that follow, and the details for the methods used in those studies are provided in the Examples.

In Vitro Study Results

The present invention has evaluated the in vitro cytoprotective effect of HSCF extracts on oxidative stress-mediated cell damage by using hepatocyte-derived cell line, HepG2 cells. In this experiment, any cytotoxic effect of HSCF extracts itself was firstly observed in HepG2 cells using MTT (3-(4,5-dimethylthiazol-2-yl)-2,5-diphenyltetrazolium Bromide) assay. Next, cytoprotective effect of sub-lethal dose of HSCF extracts was evaluated by using tert-butyl hydroperoxide (tBHP)-induced cellular damage model. To examine antioxidant effect of HSCF extracts, the present invention observed whether Nrf2, master transcription factor of antioxidant genes, was transactivated by HSCF extracts. Finally, antioxidant capacity by HSCF extracts was monitored based on superoxide dismutase (SOD) and catalase (CAT) activities, and expression of antioxidant genes—glutamate-cysteine ligase catalytic subunit (GCLC), hemeoxygenase-1 (HO1) and NAD(P)H dehydrogenase quinone 1 (NQO1).

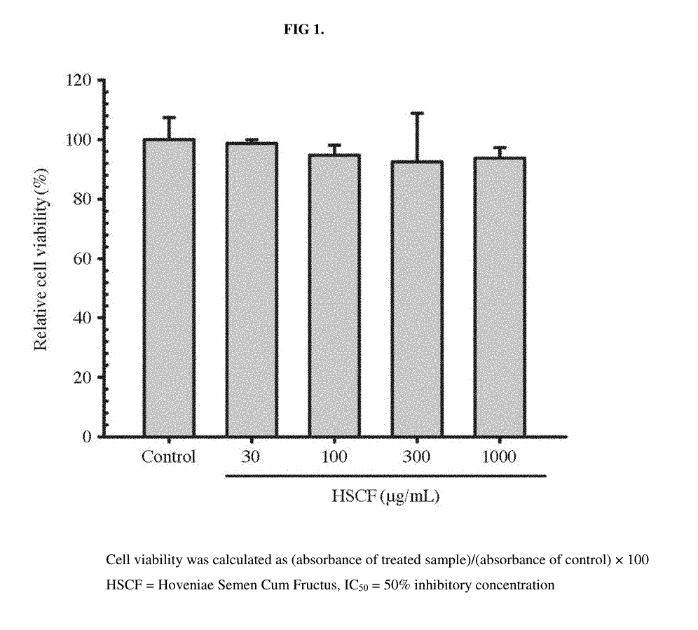

Prior to examining the role of HSCF extracts on oxidative stress and antioxidant gene induction, the cytotoxic effects of HSCF extracts itself in HepG2, hepatocyte-derived cells were first monitored. HepG2 cells were plated at a density of 5×10⁴ cells per well in a 24-well plate. After serum starvation for 12 hrs, HepG2 cells were further incubated with 30, 100, 300, or 1000 g/ml HSCF extracts for 24 hrs and then analyzed cell viability by MTT assay calculated as (absorbance of treated sample)/(absorbance of control)×100. Result from MTT assay indicated that HSCF extracts up to 1000 g/ml concentration did not affect the viability of HepG2 cells. The relative cell viabilities of 30, 100, 300 and 1000 g/ml HSCF extracts treatments were 96.72±1.22, 94.75±3.35, 92.49±16.31, and 93.78±3.49%, respectively (FIG. 1). IC₅₀ of HSCF extracts in HepG2 cells seemed to be above 1000 g/ml. Therefore, 30-1000 g/ml HSCF extracts were used for the subsequent experiments.

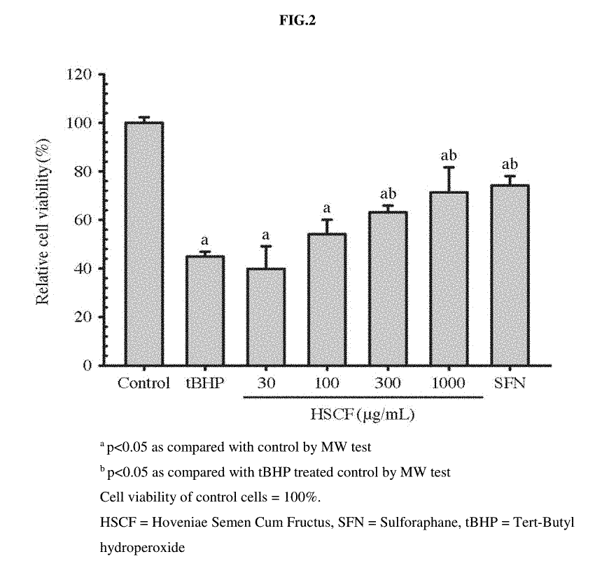

Next, the effects of HSCF extracts on tBHP-induced cytotoxicity were examined. tBHP, an analog of lipid hydroperoxide, is frequently used as an oxidative stress inducer to screen antioxidant drug candidate [Cooke et al., 2002]³⁰. Cells were pretreated with sublethal dose of HSCF extracts (30-1000 g/ml) and then further incubated with 150 M of tBHP for 24 hrs. MTT assay was conducted to test whether or not HSCF extracts prevented tBHP-induced cytotoxicity. The results showed that cells exposed to 150 M tBHP significantly reduced about 55% in cell viability compared to the control, and SFN (30 M) pretreatment significantly protected from tBHP-mediated cell death. However, HSCF extracts pre-treatment reversed the effects of tBHP in HepG2 cells by increasing cell viability in a concentration-dependent manner. The significant cytoprotective effect was observed at 300 or 1000 g/ml of HSCF extracts treatment. The relative cell viabilities of 30, 100, 300 and 1000 g/ml HSCF extracts in tBHP-treated cells were 39.86±9.27, 54.15±5.92, 64.10±2.83, and 71.36±10.30%, respectively (FIG. 2).

Nrf2 is an essential transcription factor that protects cells against oxidative stress and enhances cellular defense system through induction of antioxidant genes [Kobayashi and Yamamoto, 2005; Lee et al., 2005]. Activated Nrf2 is released from its cytosolic repressor Keap1, translocates into the nucleus, binds to ARE in the promoter regions, and then induces the expression of antioxidant genes [Itoh et al., 2004, Ishii et al., 2002]. Therefore, ARE activation is crucial for enhancing antioxidant capacity of cells.

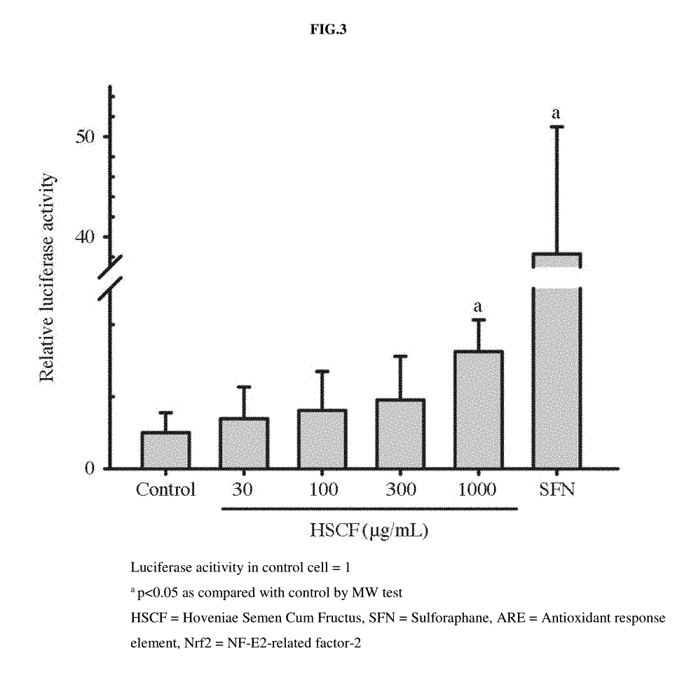

To examine the possibility that ARE activation by Nrf2 is associated with HSCF extracts-mediated cytoprotection, pGL4.37 tranfected HepG2 cells (5×10⁵ cells/well) were plated in 12-well overnight, serum starved for 12 hrs, and then subsequently exposed to 30-1000 μg/ml HSCF extracts for 18 hrs. ARE-driven luciferase activity was measured in the cell treated with HSCF extracts. As expected, SFN (30 M) significantly increased luciferase activity. 30-1000 g/ml HSCF extracts treatment gradually increased the luciferase activity in a concentration dependent manner. The significant increase in ARE-luciferase activity was observed in 1000 g/ml HSCF extracts treatment. The relative ARE-luciferase activities were 1.39±0.88, 1.62±1.08, 1.91±1.22, and 3.25±0.88 in 30, 100, 300, and 1000 g/ml HSCF extracts treated cells, respectively (FIG. 3).

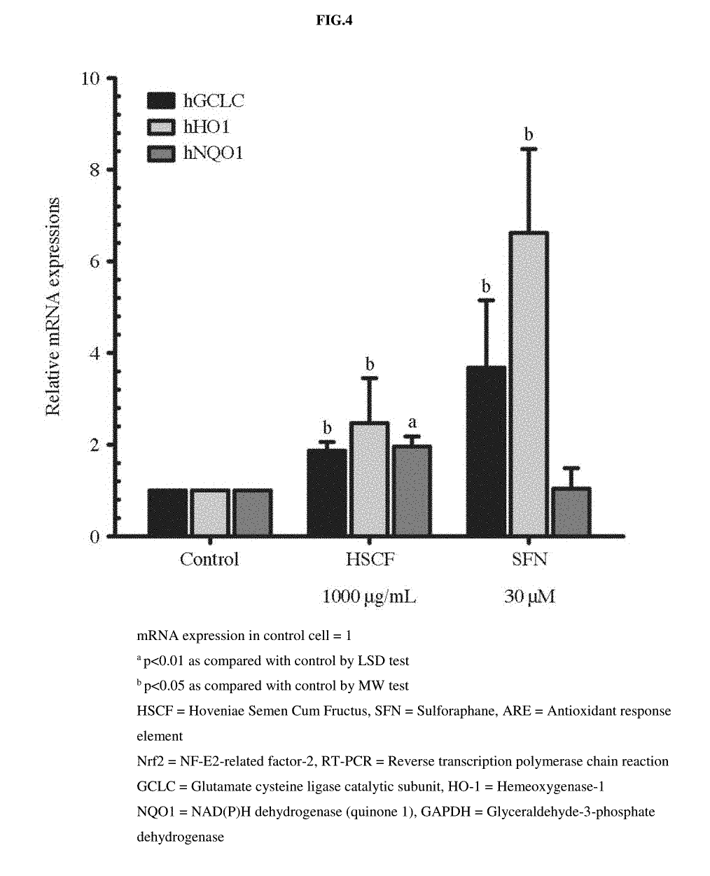

Next, the effects of HSCF extracts on the mRNA expression of antioxidant genes were examined. Cells were incubated with 1000 μg/ml HSCF extracts or 30 μM SFN for 12 hrs. To verify whether ARE transactivation by 1000 g/ml HSCF extracts in HepG2 cells corresponds with expression of Nrf2-mediated antioxidant enzymes, such as GCLC, HO1, and NQO1, realtime RT-PCR analysis was conducted and showed that HSCF extracts significantly increased mRNA levels of the antioxidant enzymes. The relative GCLC, HO1, and NQO1 mRNA levels in 1000 g/ml HSCF extracts treated cells were 1.87±0.19, 2.47±0.98, and 1.95±0.22, respectively (FIG. 4). Expression of Nrf2-mediated target gene promotes cell survival in oxidizing environments via enhancement of free radical metabolism, regulation of proteasome function, maintenance of glutathione homeostasis, inhibition of cytokine-mediated inflammation, and recognition of damaged DNA [Kensler et al., 2007]. HO1 is a highly inducible enzyme that catalyzes the rate-limiting step of free heme degradation into Fe²⁺, carbon monoxide, and biliverdin [Abraham and Kappa, 2008]. There is increasing evidence to suggest that induction of HO-1 protects against a variety of chronic disease. GCLC, a heterodimeric protein comprising catalytic and modifier subunits, plays an essential role in maintaining cellular redox homeostasis and reducing oxidative damage by glutathione synthesis [Franklin et al., 2009]. It has been reported that a variety of cellular signaling pathways such as p38 MAPK, ERK, PI3K, PKC or casein kinase are involved in activation of Nrf2 [Kensler et al., 2007; Zhang et al., 2008, Apopa et al., 2008; Rushworth et al., 2006; Zimmermann et al., 2015]^{34,37,38,39,40}. Recently, it was proposed that H. dulcis increase AMP-activated protein kinase (AMPK) phosphorylation in adipocytes [Kim et al., 2014]¹⁸. AMPK is activated in response to metabolic stress and plays a role in compensatory responses that protect cells from stress [Steinberg and Kemp, 2009]. It was reported that AMPK activation sensitizes Nrf2/HO1 signaling cascade [Zimmermann et al., 2015]. Therefore, AMPK activation by HSCF extracts would be one of plausible mechanisms for Nrf2 activation and its target genes induction.

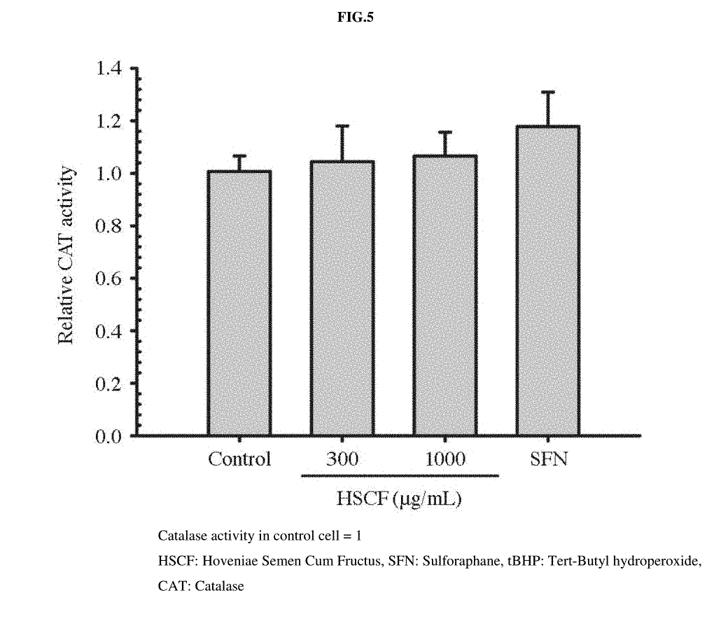

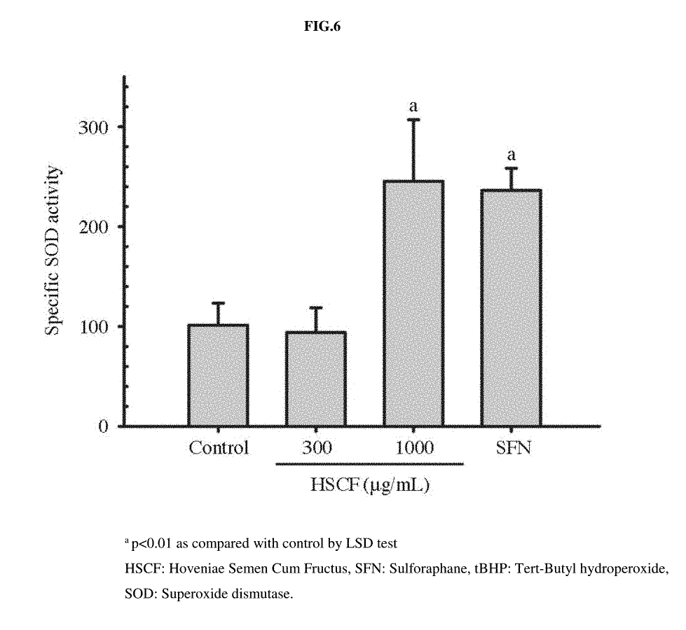

It has been reported that tBHP causes apoptotic death in hepatocytes through ROS production, GSH reduction and dysfunction of mitochondrial membrane permeability [Moon et al., 2014]. Antioxidant enzyme such as CAT, and SOD are essential in both scavenging ROS and maintaining cellular stability [Somani, 1996]. Catalase is an enzyme that catalyzes the conversion of H₂O₂ to H₂O, and SOD is one the antioxidant enzyme that diminishes ROS by conversion of superoxide radical to H₂O₂. To examine whether hepatoprotection of HSCF extracts by tBHP is associated with enhancement of antioxidant capacity, activities of antioxidant enzymes by HSCF extracts treatment were measured. Although 300 and 1000 g/ml HSCF extracts treatment tended to slightly increase in 4.46% and 6.55% O₂-foam formation by catalase, those increases were not statistically significant (FIG. 5). Significant increases of SOD activity were observed only in 1000 g/ml HSCF extracts treatment as compared with control cells. Specific SOD activities in 300 and 1000 g/ml HSCF extracts treated cells were 94.01±24.71 and 245.41±61.68 U/mg protein, respectively (FIG. 6). Therefore, increases in antioxidant defense system by HSCF extracts may be correlated with protective effect of HSCF extracts against oxidative stress.

Taken together the results of this experiment, the present invention has found that 300 or 1000 g/ml of HSCF extracts have favorable hepatoprotective effects against oxidative stress through Nrf2-mediated antioxidant gene induction. HSCF extracts 300 or 1000 g/ml showed dose-dependent hepatoprotective effects against tBHP in HepG2 cells. As a plausible molecular mechanism of HSCF extracts, 1000 g/ml HSCF extracts treatment significantly increased the Nrf2 transactivation and induced its target genes expression. Furthermore, 1000 g/ml HSCF extracts enhanced SOD activity in this in vitro study.

In Vivo Study Results

More systemic evaluation of the hepatoprotective effects of HSCF extract with molecular targets was needed. As such, the present invnetion also conducted an in vivo study to systemically evaluate the beneficial potential of HSCF extract on the subacute EtOH-induced hepatic damages in mice as well as the corresponding potent anti-oxidant, anti-inflammatory and anti-steatosis mechanisms.

As an initial matter, the body weight decrease after EtOH treatment was considered a result of the direct toxicity of EtOH and/or indirect toxicity related to liver damage. The body weight can also decrease due to malnutrition, secondary to food intake decreases [Saravanan and Nalini, 2007; Saravanan et al., 2007]. Therefore, the increased body weight and gains detected in the silymarin group and HSCF extracts treated group are indirect evidence of hepatoprotective effects as compared with the EtOH control, since body weight is considered a putative indicator of health. In addition, dose-dependent inhibitory effects on the EtOH-induced liver weight decreases following treatment with HSCF extracts are also evidence that HSCF extracts have hepatoprotective effects against acute EtOH intoxication.

In chronic alcoholics, the liver weight is generally decreased due to necrotic and inflammatory processes that occur in the hepatic parenchyma and the substitution of hepatic parenchyma with lipids 63-66. This is also the case in acute EtOH-induced liver damaged mice [Park, 2013]. HSCF extracts at 500 mg/kg showed similar inhibitory effects on the EtOH-induced liver weight decreases as compared with silymarin at 250 mg/kg in this experiment.

Generally, AST, ALT, albumin, γ-GTP and ALP are used as serum markers to represent various types of liver damage [Sodikoff, 1995]. These markers were markedly elevated following EtOH-induced hepatic damages in previous reports [Li et al., 2004; Das et al., 2009], and also in this experiment. In addition, serum TG levels are generally increased with EtOH-induced hepatic damage due to decreased TG utilization in hepatocytes [Ho et al., 2012; Xiang et al., 2012]. Therefore, it is considered evidence that HSCF extracts have favorable hepatoprotective effects against EtOH-induced liver injuries because marked inhibition of the EtOH-induced serum AST, ALT, albumin, ALP, γ-GTP and TG levels, and hepatic TG contents were dose-dependently improved in these groups as compared with the EtOH control mice in the present study. HSCF extracts at 500 mg/kg showed similar inhibitory effects on the EtOH-induced serum AST, ALT, albumin, ALP, TG and γ-GTP elevation as compared with silymarin at 250 mg/kg.

Abnormal metabolism of cytokines, especially TNF-α, is another major feature of alcoholic liver disease [Song et al., 2006; Xing et al., 2011]. It was initially observed that cultured monocytes from alcoholic hepatitis patients spontaneously produced TNF-α and produced significantly more TNF-α in response to a lipopolysaccaride stimulus than control monocytes [McClain and Cohen, 1989]. Subsequently, earlier researchers demonstrated that anti-TNF antibody prevented liver injury in alcohol-fed rats and mice lacking the TNF-type I receptor also did not develop alcoholic liver injury [Iimuro et al., 1997; Yin et al., 1999]. Consistent with chronic alcohol effects, increased hepatic TNF-α production by acute EtOH exposure has recently been reported [Zhou et al., 2003; Song et al., 2006; Xing et al., 2011]. In vitro studies demonstrated that silymarin inhibited Kupffer cell functions and TNF-α production in lipopolysaccaride-stimulated RAW264.7 cells [Dehmlow et al., 1996; Cho et al., 2000]. Our results also showed that 2 weeks of continuous subacute EtOH administration enhanced hepatic TNF-α production. In vivo HSCF extracts administration dose-dependently attenuated this increased TNF-α production, similar to silymarin at 250 mg/kg, suggesting that the hepatoprotective effects of HSCF extracts on EtOH-induced subacute hepatic damages may be mediated by anti-inflammatory effects through suppression of the hepatic TNF-α production.

Although there are many potential sources of ROS in response to EtOH exposure, CYP450 2E1 is one of the major sites involved in ROS production in the liver in response to alcohol [Song et al., 2006]. It has been reported that long-term alcohol exposure increased CYP450 2E1 activities [Lieber, 1997; Zhou et al., 2002]. Furthermore, investigations using CYP450 2E1 inhibitors, including diallyl sulfide or chlormethiazole, have shown that inhibition of CYP450 2E1 activity inhibits alcohol-induced liver injury, indicating the importance of CYP450 2E1 in alcohol-induced ROS accumulation and liver injury [Gouillon et al., 2000; McCarty, 2001]. Similarly, genetic overexpression of CYP450 2E1 in the liver causes enhanced alcohol-induced liver injury in mice [Morgan et al., 2002]. To investigate the possible mechanisms by which HSCF extracts attenuated subacute EtOH-induced liver injury, we first evaluated the effect of HSCF extracts on CYP450 2E1 enzymatic activity in response to acute EtOH exposure. Our study indicated that 2 weeks of continuous oral administration of EtOH increased hepatic CYP450 2E1 activity, but this increase of CYP450 2E1 activity was dose-dependently diminished by treatment with HSCF extracts. HSCF extracts at 500 mg/kg showed similar inhibitory effects on the EtOH-induced hepatic CYP450 2E1 activity increases as compared with silymarin at 250 mg/kg.

Considerable experimental and clinical evidence has contributed to support a key role of oxidative stress in the pathophysiological processes of liver injury related to excessive alcohol consumption [Cahill et al., 2002; Castilla et al., 2004]. The metabolism of EtOH gives rise to the generation of excess amounts of ROS and has a detrimental effect on the cellular antioxidant defense system [Navasumrit et al., 2000; Ozaras et al., 2003] that leads to hepatic cellular necrosis, inflammation and steatohepatitis [Husain et al., 2001; Kasdallah-Grissa et al., 2007]. Thus, numerous interventions have been put forward to counteract the vulnerability of the liver to oxidative challenges during alcohol consumption by reinforcing the endogenous antioxidant defense system [Koch et al., 2000; Ozaras et al., 2003]. Lipid peroxidation is an autocatalytic mechanism leading to oxidative destruction of cellular membranes [Videla, 2000; Subudhi et al., 2008]. Such destruction can lead to cell death and to the production of toxic and reactive aldehyde metabolites called free radicals, with MDA as the most important [Venditti and Di, 2006; Messarah et al., 2010]. It is known that ROS leads to oxidative damage of biological macromolecules, including lipids, proteins, and DNA [Das and Chainy, 2001; Messarah et al., 2010], and oxidative stress influences body adipocytes, resulting in decreases in body fat mass and related body weight decreases [Voldstedlund et al., 1995]. MDA is a terminal product of lipid peroxidation. So the content of MDA can be used to estimate the extent of lipid peroxidation [Messarah et al., 2010]. Marked increases of liver MDA contents have been observed in alcoholic rodents [Kasdallah-Grissa et al., 2007; Song et al., 2006; Xing et al., 2011; Wang et al., 2013; Yang et al., 2013], and liver MDA content was increased in this study by treatment with EtOH. GSH is a representative endogenous antioxidant that prevents tissue damage by keeping the ROS at low levels and at certain cellular concentrations, and is accepted as a protective antioxidant factor in tissues [Odabasoglu et al., 2006]. SOD is one of the antioxidant enzymes that contributes to enzymatic defense mechanisms, and catalase is an enzyme that catalyzes the conversion of H₂O₂ to H₂O [Cheeseman and Slater, 1993]. The decrease of antioxidant enzyme activities such as SOD and catalase, and GSH contents may be indicative of the failure to compensate for the oxidative stress induced by EtOH [Navasumrit et al., 2000; Husain et al., 2001; Ozaras et al., 2003; Kasdallah-Grissa et al., 2007]. In this experiment, the hepatic antioxidant defense system was dose-dependently enhanced by treatment with HSCF extracts at 500, 250 and 125 mg/kg as compared with the EtOH control, along with up-regulation of Nrf2, a master transcription factor of antioxidant genes [Kobayashi and Yamamoto, 2005; Lee et al., 2005], which was down-regulated by EtOH supply. HSCF extracts at 500 mg/kg showed similar inhibitory effects on the EtOH-induced hepatic lipid peroxidation, and enhancement effects on the hepatic endogenous antioxidant defense systems as compared with silymarin at 250 mg/kg. This suggests that the hepatoprotective effects of HSCF extracts against EtOH intoxication are mediated by augmentation of the hepatic antioxidant defense system, which may be mediated by Nrf2 activation and related inhibitory effects on lipid peroxidation.

There are multiple mechanisms underlying EtOH-induced development of fatty liver. Enhanced lipogenesis and impaired fatty-acid oxidation have long been proposed as important biochemical mechanisms underlying the development of alcoholic fatty liver [Wang et al., 2013; Yang et al., 2013]. Previous studies demonstrated that EtOH administration activates SREBP-1c and its target genes like SCD1, ACC1 and FAS, which promote de novo fatty-acid synthesis [Wada et al., 2008; Wang et al., 2013; Yang et al., 2013]. SREBP-1c-null mice fed EtOH by intragastric infusion for 4 weeks showed significantly lower TG concentration than that in wild typed mice [Ji et al., 2006]. In this experiment, EtOH treatment also significantly up-regulated the hepatic SREBP-1c mRNA expression, and its target genes—FAS, SCD1 and ACC1. However, all dosages of HSCF extracts dose-dependently down regulated the hepatic mRNA expression of SREBP-1c, SCD1, ACC1 and FAS. This suggests that the hepatoprotective effects of HSCF extracts against EtOH-induced hepatic steatosis are mediated by down regulation of SREBP-1c and its target genes, FAS, SCD1 and ACC1. HSCF extracts at 500 mg/kg showed similar inhibitory effects on the EtOH-induced increases of hepatic lipogenic genes—SREBP-1c, FAS, SCD1 and ACC1 mRNA expression as compared with silymarin at 250 mg/kg.

PPARγ and DGAT2 are involved in TG synthesis [Herzig et al., 2003; Yu et al., 2003; Wada et al., 2008; Wang et al., 2013; Yang et al., 2013]. DGAT is involved in TG synthesis in the liver, and the levels of DGAT1 and DGAT2 mRNAs were increased in response to EtOH [Wang et al., 2013; Yang et al., 2013]. PPARγ is a member of the nuclear receptor superfamily of ligand-activated transcription factor that regulate the expression of genes associated with lipid metabolism. Adenovirus-mediated delivery of PPARγ to hepatocytes leads to fatty liver, and PPARγ RNA interference is reported to decrease hepatic TG levels [Yu et al., 2003; Herzig et al., 2003]. PPARγ and DGAT are significantly up-regulated after acute EtOH administration and are involved in EtOH-induced fatty liver in mouse [Wada et al., 2008; Wang et al., 2013; Yang et al., 2013]. In this study, hepatic mRNA levels of both PPARγ and DGAT2 were up-regulated by EtOH stimulation. HSCF extracts at 500, 250 and 125 mg/kg significantly and dose-dependently impaired the elevation of these genes, similar to silymarin at 250 mg/kg, well corresponding to the results of hepatic and serum TG levels. These results suggested that oral treatment of HSCF extracts dose-dependently inhibits hepatic lipogenesis in response to EtOH by suppressing genes related to TG synthesis.

In addition to increased lipogenesis, decreased fatty acid metabolism also contributes to EtOH-induced fatty liver [Wada et al., 2008; Hu et al., 2013; Wang et al., 2013; Yang et al., 2013]. PPARγ and its target genes, including ACO and CPT1, are involved in fatty-acid β-oxidation [Reddy and Mannaerts, 1994; Wang et al., 2013; Yang et al., 2013]. Administration of Wy14643, a PPARγ agonist, prevented fatty liver in mice fed EtOH for 4 weeks [Crabb et al., 2004; Fischer et al., 2003]. In this experiment, subacute treatment of EtOH 5 g/kg decreased the expression of these genes and impaired fatty-acid β-oxidation in the liver. However, HSCF extracts at 500, 250 and 125 mg/kg up-regulated the hepatic mRNA expression of PPARγ and its target genes, including ACO and CPT1, similar to silymarin at 250 mg/kg. Oral treatment of HSCF extracts at dose levels of 500, 250 and 125 mg/kg not only down regulated the expression of genes related to fatty-acid and TG synthesis, but also increased fatty acid metabolism through up-regulation of genes involved in fatty-acid β-oxidation in the liver.

Acute or chronic alcohol consumption can cause severe histopathological liver injury [Dey and Cederbaum, 2006]. Alcohol is known to impair fat oxidation and to stimulate lipogenesis in the liver [You and Crabb, 2004ab; Donohue, 2007]. Thus, alcohol consumption can lead to the development of hepatic steatosis [Chen et al., 2009]. In this experiment, severe deposition of lipid droplets in the cytoplasm of hepatocytes and hepatosteatosis were also observed in all EtOH treated mice. This EtOH-induced hepatosteatosis was re-confirmed with histomorphometry based on the number of changed fatty hepatocytes, mean diameters of hepatocytes and percentages of changed fatty regions, which were significantly increased in EtOH control mice as compared with intact control mice in the left lateral lobes. However, this EtOH treatment-related histopathological hepatosteatosis was significantly and dose-dependently inhibited by treatment of HSCF extracts at 500, 250 and 125 mg/kg, similar to silymarin 250 mg/kg, as compared with EtOH control mice in this experiment. These findings are considered direct evidence that HSCF extracts have favorable hepatoprotective effects against EtOH-induced hepatic steatosis.

NT is a product of tyrosine nitration mediated by reactive nitrogen species such as peroxynitrite anion and nitrogen dioxide. It is detected in a large number of pathological conditions including EtOH-induced liver damages, and is considered a marker of nitric oxide-dependent, reactive nitrogen species-induced nitrative stress [Chen et al., 2004; Mohiuddin et al., 2006; Pacher et al., 2007]. Most studies on alcoholic hepatic steatosis have focused on the ability of EtOH to shift the redox state in the liver and to inhibit fatty acid oxidation [Donohue, 2007; Rogers et al., 2008]. Indeed, previous studies have shown the repression of some enzymes involved in fatty acid oxidation and induction of lipogenic enzymes in EtOH-fed animals [You and Crabb, 2004ab]. Sustained exposure to ROS leads to prolonged oxidative stress and increases of NT [Zhou et al., 2005; Leung et al., 2012]. In this experiment, marked and significant increases of NT-immunoreactive cells were observed in the hepatic tissues of EtOH control mice as compared with intact control mice, but they were significantly reduced by treatment of HSCF extracts at 500, 250 and 125 mg/kg, dose-dependently, similar to silymarin at 250 mg/kg. It is suggested that HSCF extracts favorably inhibit iNOS related oxidative stress and protect against hepatocyte necrotic changes from EtOH at dose levels of 500, 250 and 125 mg/kg.

4-HNE is an α, β-unsaturated hydroxyalkenal which is produced by lipid peroxidation in cells. It is considered a possible causal agent of numerous diseases, such as chronic inflammation, neurodegenerative diseases, adult respiratory distress syndrome, atherogenesis, diabetes and different types of cancer [Zarkovic, 2003; Dubinina and Dadali, 2010; Smathers et al., 2011]. Sustained exposure to EtOH mediated ROS leads to prolonged oxidative stress, which promotes lipid peroxidation and generation of reactive aldehydes, such as 4-HNE [Galligan et al., 2012; Leung et al., 2012]. In the present study, marked and significant increases of 4-HNE-positive cells were also observed in the left lateral hepatic lobes of EtOH control mice as compared with intact control mice, but they were significantly and dose-dependently normalized by treatment of all dosages of HSCF extracts, similar to silymarin at 250 mg/kg. This corresponded to the results of NT-immunolabeled cells and is considered as direct evidence that HSCF extracts effectively inhibited lipid peroxidation and the formation of 4-HNE to protect against hepatocyte necrotic changes from EtOH.

Results corresponding to previous reports [Song et al., 2006; Xing et al., 2011; Wang et al., 2013; Yang et al., 2013] regarding marked decreases of body and liver weights, increases of serum AST, ALT, Albumin, γ-GTP and TG levels, hepatic TG contents, TNF-α level, CYP450 2E1 activity and mRNA expression of hepatic lipogenic genes (SREBP-1c, SCD1, ACC1, FAS, PPARγ and DGAT2), decreases mRNA expression of genes involved in fatty acid oxidation (PPARα, ACO and CPT1) or master transcription factor of antioxidant gene (Nrf2) were observed with histopathological changes related to hepatosteatosis (noticeable increases of the percentages of changed fatty regions, the number of changed fatty hepatocytes and mean hepatocyte diameters) and increases of NT and 4-HNE-immunolabelled hepatocytes, following continuous oral administration of EtOH for 2 weeks in the present study. Also, the destruction of hepatic antioxidant defense systems (the increase of hepatic lipid peroxidation, increase of liver MDA contents, and decreases of GSH contents, SOD and CAT activities) were demonstrated in EtOH control mice as compared with intact control. However, these EtOH treatment related liver inflammatory damages, steatosis, increases of mRNA expression of hepatic lipogenic genes, decreases of mRNA expression of genes involved in fatty acid oxidation, and destruction of antioxidant defense systems, may be mediated by down-regulation of Nrf2, which was markedly and dose-dependently inhibited by pretreatment of HSCF extracts at 500, 250 and 125 mg/kg. The overall effects of HSCF extracts at 500 mg/kg were similar to those of silymarin at 250 mg/kg in this experiment.

### EXAMPLES

### Example 1

### In Vitro Study

Test Materials

The HSCF extracts as a beige-colored (off-white) powder were supplied by Aribio (Seoul, Korea). It was well suspended up to 50 mg/ml concentration in distilled water. Some specimens of HSCF extracts were deposited in the herbarium of the Medical Research center for Globalization of Herbal Formulation, Daegu Haany University (Code HSCF2014Ku). Clear liquid of SFN (Sulforaphane Sigma-Aldrich, St. Louise, Mo., USA) was used as standard reference drug at 30 μM levels. All test materials were stored at −20° C. in a refrigerator to protect from light and humidity until used.

Cell Culture

HepG2 cells, a human hepatocyte-derived cell line, were purchased from American Type Culture Collection (ATCC, Rockville, Md., USA). The cells were plated at 1×10⁵ per well in 6-well plates, and wells with 70-80% confluency were used. Cells were cultured in DMEM containing 10% fetal bovine serum with 100 units/ml penicillin/streptomycin at 37° C. in a humidified atmosphere with 5% CO₂.

MTT Assay

The cells were plated at a density of 5×10⁴ cells per well in a 24-well plate to determine cytoprotective activity of HSCF extracts. Cells were serum-starved for 12 hrs, and then treated HSCF extracts 1 hr prior to the addition of 150 M tBHP and the cells were further incubated for 12-24 hrs. After incubation of the cells, viable cells were stained with MTT (0.1 μg/mL, 4 hrs; Sigma-Aldrich, St. Louise, Mo., USA). The media were then removed, and produced formazan crystals in the wells were dissolved by addition of 300 μL of DMSO per well. To compare cytoprotective effect of herbal extract, 30 μM of sulforaphane was used as positive control. Absorbance was measured at 570 nm using a Titertek Multiskan automatic multimode reader (Model Infinite 200 PRO; Tecan, Männedorf, Switzerland). Cell viability was defined relative to control cells as [viability (% of control)=(absorbance of treated sample)/(absorbance of control)×100].

Reporter Gene Assays

ARE-driven reporter gene construct, pGL4.37 [luc2P/ARE/Hygro] was obtained from promega (Madison, Wis., USA). HepG2 cells were stably transfected with pGL4.37 plasmid using Fugene HD (Promega, Madison, Wis., USA) according to manufacturer's instruction and 80 g/mL hygromycin was added to select the resistant colonies. The resistant colonies were pooled and used for reporter gene analysis. To determine luciferase activity, stably transfected cells (5×10⁵ cells/well) were replated in 12-well plates overnight, serum starved for 12 hrs, and exposed to the HSCF extracts for 24 hrs. Luciferase activities in cell lysates were measured by adding luciferase assay reagent (Promega, Madison, Wis., USA) using Titertek Multiskan automatic multimode reader (Model Infinite 200 PRO; Tecan, Männedorf, Switzerland). The relative luciferase activity was calculated as the relative change to protein content determined by bicinchoninic acid (BCA) method.

Measurements of CAT and SOD Enzyme Activities

Measurement of CAT activity was accomplished according to Iwase et al. [2013]. In brief, cells were placed at 100 pi dish (8×10⁶cells/dish) overnight, followed by serum-starved for 12 hrs. Then cells were exposed to the HSCF extracts or 30 M of sulforaphane as positive control for 12 hrs. After treatment, cells were scraped by phosphate buffered saline and a quantity of cells was counted for further calculation. Same volume of cell suspension, Triton X-100 (Sigma-Aldrich, St. Louis, Mo., USA), H₂O₂ (Sigma-Aldrich, St. Louis, Mo., USA) were added to glass tube (12×75 mm) in order. Mixture was incubated at room temperature for 15 min. After O₂-foaming reaction ends completely, height of O₂-foam was measured by ruler. Catalase activity was calculated by relative change of O₂-foam's height to counted cell number. The activity of SOD was determined using a commercial test kit (Cayman Chemical, Ann Arbor, Mich., USA) which utilizes a tetrazolium salt for detection of superoxide radicals generated by xanthine oxidase and hypoxanthine at 550 nm. One unit (U) of SOD is defined as the amount of enzyme needed to exhibit 50% dismutation of the superoxide radical.

Preparation of RNA and Real-Time PCR Assays

Total RNA was isolated from treated cells by using Trizol reagent (Invitrogen, Carlsbad, Calif., USA). The RNA (2 g each) was reverse-transcribed using oligo-d(T)₁₆ primers to obtain cDNA. PCR was performed using the human specific primers for HO-1 (sense: CAGGAGCTGCTGACCCATGA, antisense: AGCAACTGTCGCCACCAGAA, product size: 195 bp), GCLC (sense: GAAGTGGATGTGG ACACCAG, antisense: TTGTAGTCAGGATGGTTTGCGA, product size: 128 bp), NQO-1 (sense: GGAT TGGACCGAGCTGGAA, antisense: TGCAGTGAAGATGAAGGCAAC, product size: 137 bp) obtained from Bioneer (Daejon, Korea). Real-time PCR was carried out according to the manufacturer's instructions (SyBr green Ex-Taq master mix, Takara, Shiga, Japan) using CFX96™ Real-Time Thermal cycler (Bio-Rad, Hercules, Calif., USA). The relative levels of each specific antioxidant genes were normalized based on the level of glyceraldehyde-3-phosphate dehydrogenase (GAPDH). After PCR amplification, a melting curve of each amplicon was determined to verify its accuracy.

Statistical Analyses

All data were expressed as mean±SD. Multiple comparison tests for different dose groups were conducted. Variance homogeneity was examined using the Levene test [Levene, 1981]⁴⁴. If the Levene test indicated no significant deviations from variance homogeneity, the obtain data were analyzed by one way ANOVA test followed by least-significant differences multi-comparison (LSD) test to determine which pairs of group comparison were significantly different. In case of significant deviations from variance homogeneity was observed at Levene test, a non-parametric comparison test, Kruskal-Wallis H test was conducted. When a significant difference is observed in the Kruskal-Wallis H test, the Mann-Whitney U (MW) test was conducted to determine the specific pairs of group comparison, which are significantly different [Ludbrook, 1997]. Statistical analyses were conducted using SPSS for Windows (Release 14.0K, SPSS Inc., Chicago, Ill., USA)

### Example 2

### In Vivo Study

Preparations and Administration of Test Materials

HSCF extracts (contains about 8.20 ug/mg quercetin) were supplied by Aribio (Seoul, Korea) as a beige powder. HSCF was ground and extracted with hot water 2 times at 95° C. for 4 hours then filtered and condensed using a rotary vacuum evaporator (EYELA N-1200B, USA). Finally, it was dried and standardized with dextrin using a spray drier (about 7.4 ug/g quercetin). The HSCF extract was obtained as 26%. A reddish-yellow powder of silymarin was purchased from Sigma-Aldrich (St. Louise, Mo., USA) as the reference drug. All test materials were stored at −20° C. in a refrigerator to protect from light and humidity until used. In this study, 500 mg/kg was selected as the highest dose of the HSCF extract based on the clinical dosage in humans and 250 and 125 mg/kg were additionally selected as the middle and lowest doses with a common ratio of 2, respectively. HSCF extract (500, 250, and 125 mg/kg) and Silymarin (250 mg/kg) were suspended in distilled water and orally administered once a day after 1 hour of EtOH treatment for 14 days. In intact and EtOH control mice, equal volumes of distilled water were orally administered.

Animals and Experimental Design

A total of sixty-three healthy male SPF/VAF Inbred C57BL/6J mice (6-wk old upon receipt; OrientBio, Seungnam, Korea) were used after acclimatization for 10 days. Animals were allocated five per polycarbonate cage in a temperature (20-25° C.) and humidity (50-55%) controlled room. The dark light cycle was 12 hrs long. Commercial rodent feed (Samyang, Seoul, Korea) and tap water were supplied ad libitum. All animals were treated according to the international regulations for the usage and welfare of laboratory animals, and the protocol was approved by the Institutional Animal Care and Use Committee in Daegu Haany University (Gyeongsan, Gyeongbuk, Korea) Approval No DHU2014-082. Eight mice in each group, with a total of six groups, were selected based on the body weights by ascending order after acclimatization: Intact control—Isocalorical maltose solution and distilled water administered mice, EtOH control: EtOH and distilled water administered mice, Silymarin group: EtOH and silymarin 250 mg/kg as reference drug treated mice, HSCF 500: mice administered EtOH and HSCF extracts at 500 mg/kg, HSCF 250: mice administered EtOH and HSCF extracts at 250 mg/kg, HSCF 125: mice administered EtOH and HSCF extracts at 125 mg/kg.

Induction of EtOH-Mediated Subacute Hepatic Damage

Subacute EtOH-induced hepatotoxicity was induced by oral administration of EtOH (0.8 g/ml concentration; Merck, Darmstadt, Germany) at 5 g per kg once a day for 14 days, at 1 hr before oral administration of each test substance, according to previous established methods [Song et al., 2006; Wang et al., 2013; Yang et al., 2013] with some modifications. In intact control mice, isocalorical maltose solution was orally administered instead of EtOH.

Measurement of Body Weight and Liver

Changes in body weight were measured at 1 day before initial test substance administration, the day of first test substance administration, and at 1, 7, 10 and 14 days after initial HSCF or silymarin extracts administration using an automatic electronic balance (Precisa Instrument, Dietikon, Switzland). To reduce the individual differences, the body weight gains during 15 days of experiment were calculated as body weight on the last day of test substance administration—body weight on the first day of test substance administration. At sacrifice, the weights of the livers were measured (absolute wet-weights). To reduce the differences from individual body weights, relative weights were also calculated divided by body weight at sacrifice.

Measurement of Serum Biochemistry

At sacrifice, about 1 ml of venous blood was collected from the caudal vena cava under anesthesia with isoflurane (Hana Pharm. Co., Hwasung, Korea). All blood samples were centrifuged at 13,000 rpm, 4° C. for 10 min using clotting activated serum tubes. Serum AST, ALT, albumin and ALP levels were detected using a blood biochemistrical autoanalyzer (Hemagen Analyst, Hemagen Diagnostic, Columbia, Mass., USA). In addition, serum TG and γ-GTP levels were measured using another type of automated blood biochemistrical analyzer (SP-4410, Spotochem, Kyoto, Japan).

Measurement of Hepatic TG Contents and TNF-α Levels

To assess TG content, liver tissue (right lobes) was homogenized in an equal volume of normal saline and extracted with a mixture of chloroform and methanol (2:1) as described previously [Butler et al., 1961]. Zeolite (Sigma-Aldrich, St. Louise, Mo., USA) was added to remove phospholipids. The resulting extract was dried under nitrogen and dissolved in Plasmanate (1 ml; Sigma-Aldrich, St. Louise, Mo., USA). TG were measured enzymatically using commercial kits (Kyowa Medex, Tokyo, Japan) as in previous studies [Bucolo and David, 1973]. Liver samples were disintegrated in 5 volumes of ice-cold radioimmunoprecipitation assay (RIPA) buffer. After incubation on ice for 30 min, samples were centrifuged twice at 20,000×g for 15 min at 4° C. The supernatants were used for the assay. The contents of total protein were measured with the Lowry method [Lowry et al., 1951] using bovine serum albumin (Invitrogen, Carlsbad, Calif., USA). The TNF-α levels were detected by enzyme-linked immunosorbent assay (ELISA) using a murine kit (BioSource International Inc., Camarillo, Calif., USA) with a microplate reader (Tecan, Männedorf, Switzerland).

Splenic Cytokine Content Measurements

Splenic concentrations of TNF-α, IL-1β, and IL-10 were measured with a mouse TNF-α ELISA kit (BD Biosciences/Pharmingen, San Jose, Calif., USA), mouse IL-1β ELISA kit (Genzyme, Westborough, Mass., USA) and mouse IL-10 ELISA kit (Genzyme, Westborough, Mass., USA), respectively [Hotchkiss et al., 1995; Yoon et al., 2010; Park et al., 2014]. Approximately 10-15 mg of tissue samples were homogenized in a tissue grinder containing 1 ml of lysis buffer (PBS containing 2 mM PMSF and lmg/ml of aprotinin, leupeptin, and pepstatin A) [Clark et al., 1991]. Analysis was performed with 100 ml of standard (diluted in lysis buffer) or 10, 50, or 100 ml of tissue homogenate. Each sample was run in duplicate, and a portion of the sample was analyzed for protein.

Determination of CYP450 2E1 Activity

Hydroxylation of p-nitrophenol to 4-nitrocatechol, a reaction catalyzed specifically by CYP2E1, was determined colorimetrically [Song et al., 2006]. Liver tissue was homogenized in 0.15 KCl and was spun at 10,000×g for 30 min. Microsomes were isolated by further centrifugation at 105,000×g for 60 mins. For the assay, 300 ml of microsomal protein was incubated for 5 mins at 37° C., and absorbance at 535 nm was measured with 4-nitrocatechol (Sigma-Aldrich, St. Louise, Mo., USA) as a standard using a UV/Vis spectrometer (OPTIZEN POP, Mecasys, Daejeon, Korea).

Measurement of Liver Lipid Peroxidation

Liver tissues were weighed and homogenized in ice-cold 0.01M Tris-HCl (pH 7.4), and then centrifuged at 12,000×g for 15 mins as described by Kavutcu et al [1996]. The concentrations of liver lipid peroxidation were determined by estimating MDA using the thiobarbituric acid test at the absorbance of 525 nm and represented by nM of MDA/mg protein [Jamall and Smith, 1985]. Total protein was measured by the Lowry method [Lowry et al., 1951].

Measurement of Hepatic Antioxidant Defense Systems

Prepared homogenates were mixed with 0.1 ml of 25% trichloroacetic acid (Merck, West Point, Calif., USA), and then centrifuged at 4,200 rpm for 40 min at 4° C. Glutathione (GSH) contents were measured at the absorbance of 412 nm using 2-nitrobenzoic acid (Sigma-Aldrich, St. Louise, Mo., USA) [Sedlak and Lindsay, 1968]. Decomposition of H₂O₂ in the presence of catalase was performed at 240 nm [Aebi et al., 1974]. Catalase activity was defined as the amount of enzyme required to decompose 1 nM of H₂O₂ per minute, at 25° C. and pH 7.8. Measurements of SOD activities were made according to Sun et al. [1988].

Reverse Transcription-Quantitative Polymerase Chain Reaction (RT-qPCR)

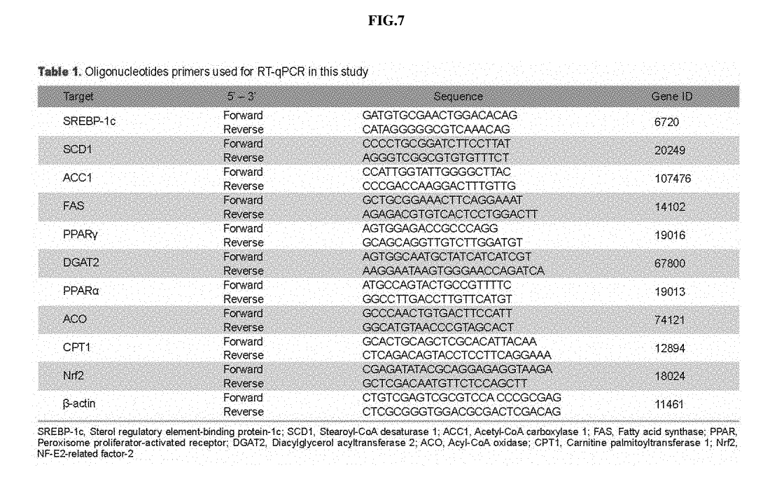

RNA was extracted using Trizol reagent (Invitrogen, Carlsbad, Calif., USA), according to the method described in previous studies [Wang et al., 2013; Yang et al., 2013]. The RNA concentrations and quality were determined with a CFX96™ Real-Time System (Bio-Rad, Hercules, Calif., USA). To remove contaminating DNA, samples were treated with recombinant DNase I (DNA-free; Ambion, Austin, Tex., USA). RNA was reverse transcribed using the reagent High-Capacity cDNA Reverse Transcription Kit (Applied Biosystems, Foster City, Calif., USA) according to the manufacturer's instructions. Briefly, the cDNA strand was first synthesized from the total RNA and then the mixture of the primers and the cDNA products was amplified by PCR. The conditions of PCR amplification were 58° C. for 30 mins, 94° C. for 2 mins, 35 cycles of 94° C. for 15 sec, 60° C. for 30 sec, 68° C. for 1 min, and then 72° C. for 5 mins. Finally, the PCR products were separated on 0.8% agarose gel. Analysis was carried out using a gel imaging system (Bio-Rad, Hercules, Calif., USA). Expression levels of SREBP-1c, SCD1, ACC1, FAS, PPARγ, DGAT2, PPARα, ACO, CPT1 and Nrf2 were calculated as a percentage relative to the intact group using β-actin RNA as the internal control. The sequences of the PCR oligonucleotide primers are listed in Table 1 as shown in FIG. 7.

Histopathological Analysis

Left lateral lobes of the liver were fixed in 10% neutral buffered formalin (NBF), and embedded in paraffin, sectioned (3-4 μm) and stained with Hematoxylin and eosin (H&E). Afterward, the histopathological profiles of each sample were observed under light microscope (Model 80i, Nikkon, Tokyo, Japan). For more detailed study, the number of hepatocytes, which occupied over 20% of lipid droplets in the cytoplasm, was calculated using an automated image analyzer (iSolution FL ver 9.1, IMT i-solution Inc., Vancouver, Canada). The value was reported as cells/1000 hepatocytes. The percentage of changed fatty regions (%/mm² of hepatic parenchyma) and the mean diameters of hepatocytes (μm/hepatocytes), with at least 10 hepatocytes per view field in the liver, were also calculated using an automated image analyzer in both the lateral and median lobes, according to the previously established method [Jung et al., 2011]. The histopathologist was blinded to the group distribution when this analysis was conducted.

Immunohistochemistry

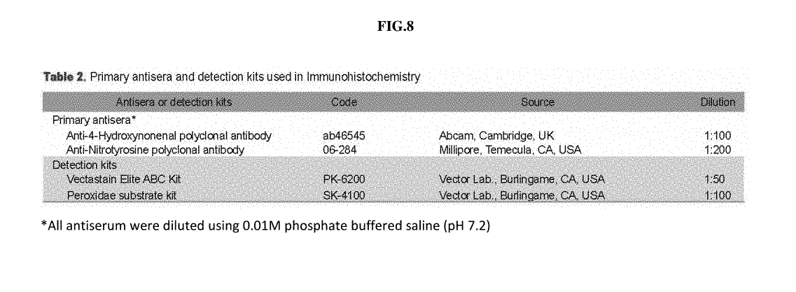

After deparaffinization of the prepared hepatic histological paraffin sections, citrate buffer antigen (epitope) retrieval pretreatment was conducted as previously described [Shi et al., 1993; Ki et al., 2013]. Briefly, a water bath with staining dish containing 10 mM citrate buffer (pH 6.0) was preheated until the temperature reached 95-100° C. The slides were immersed in the staining dish and a lid was placed loosely on the staining dish. Incubation was performed for 20 min and the water bath was turned off. The staining dish was placed at room temperature and the slides were allowed to cool for 20 minutes. After epitope retrieval, sections were immunostained using avidin-biotin complex (ABC) methods (Table 2 as shown in FIG. 8) for NT and 4-Hydroxynonenal (4-HNE) according to the previous study [Li et al., 2012; Ki et al., 2013]. Briefly, endogenous peroxidase activity was blocked by incubation in methanol and 0.3% H₂O₂ for 30 minutes, and non-specific binding of immunoglobulin was blocked with normal horse serum blocking solution (Vector Lab., Burlingame, Calif., USA. Dilution 1:100) for 1 hr in a humidity chamber. Primary antiserum (Table 2) was applied overnight at 4° C. in the humidity chamber, followed by incubation with biotinylated universal secondary antibody (Vector Lab., Dilution 1:50) and ABC reagents (Vectastain Elite ABC Kit, Vector Lab., Burlingame, Calif., USA; Dilution 1:50) for 1 hr at room temperature in the humidity chamber. Finally, reaction with a peroxidase substrate kit (Vector Lab., Burlingame, Calif., USA) was conducted for 3 min at room temperature. All sections were rinsed in 0.01M PBS 3 times between steps. The cells that showed stronger immunoreactivities in the cytoplasm with over 20% of the density against each antiserum as compared with intact control hepatocytes were regarded as positive immunoreactive. The numbers of NT- and 4-HNE-positive cells were measured for a total of 1000 hepatocytes using a digital image analyzer, according to previous reports [Hartley et al., 1999; Chen et al., 2004; Noyan et al., 2006]. The histopathologist was blinded to the group distribution when this analysis was performed.

Data Analysis

All numerical data were expressed as mean±standard deviation (SD) of eight mice. Multiple comparison tests for different dose groups were conducted. Variance homogeneity was examined using the Levene test [Levene, 1981]. If the Levene test indicated no significant deviations from variance homogeneity, the obtained data were analyzed by one-way ANOVA test followed by least-significant differences multi-comparison (LSD) test to determine which pairs of group comparisons were significantly different. In the event of significant deviations from variance homogeneity in the Levene test, a non-parametric comparison test, Kruskal-Wallis H test, was conducted. When a significant difference was observed in the Kruskal-Wallis H test, the Mann-Whitney U (MW) test was conducted to determine the specific pairs of group comparison, which are significantly different. Statistical analyses were conducted using SPSS for Windows (Release 14.0K, IBM SPSS Inc., Armonk, N.Y., USA) [Ludbrook, 1997]. In addition, the percent-point changes between intact vehicle and EtOH control were calculated to observe the severities of hepatic damages induced by 2 weeks of continuous oral treatment of EtOH in this study. The percent-point changes as compared with EtOH control and test substances treated mice were also calculated for understanding of the hepatoprotective effects of the test materials as in Equations 1 and 2, respectively, also according to our previous established method [Kang et al., 2014].

Percent-point Changes as Compared with Intact Vehicle Control (%)=((Data of EtOH control−Data of intact control)/Data of intact control)×100 Equation 1.

Percent-point Changes as Compared with EtOH Control (%)=((Data for test substance administered group−Data of EtOH control)/Data for EtOH control)×100. Equation 2.

Results

Changes of the Body Weights

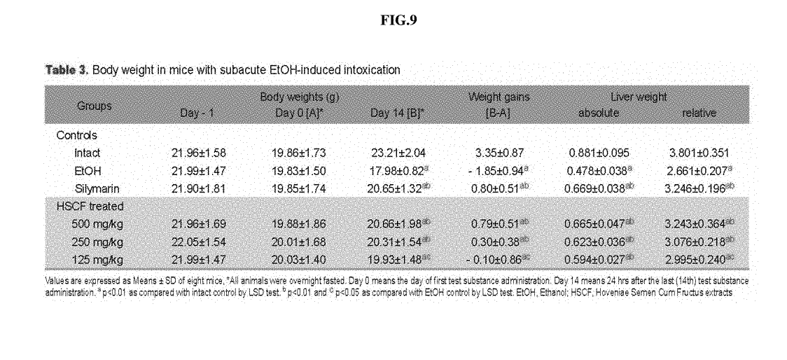

Significant (p<0.01 or p<0.05) decreases of body weight were detected from 7 days after EtOH administration in the EtOH control. The body weight gains during 15 days of experimentation were also significantly (p<0.01) decreased in the EtOH control as compared with the intact control. However, significant (p<0.01 or p<0.05) increases of body weights were observed from the 10^{th} day of test substance administration in the mice treated with HSCF extracts at 500 mg/kg and silymarin at 250 mg/kg, and from the 13^{th} day in those treated with HSCF extracts at 250 and 125 mg/kg as compared with the EtOH control. In addition, the body weight gains during 15 days of experiment were significantly (p<0.01) increased in HSCF and silymarin administered mice as compared with the EtOH control (Table 3 as shown in FIG. 9).

Changes in the Liver Weights

Significant (p<0.01) decreases of liver absolute wet and relative weights were detected in EtOH control mice as compared with the intact control. However, these EtOH-induced decreases of liver absolute and relative weights were dose-dependently and significantly (p<0.01) inhibited by treatment with HSCF extracts at 500, 250 and 125 mg/kg as compared with the EtOH control mice. HSCF extracts at 500 mg/kg showed similar inhibitory effects on the EtOH-induced liver weight decreases as compared with silymarin at 250 mg/kg in this experiment (Table 3 as shown in FIG. 9).

Changes in the Serum Biochemistry

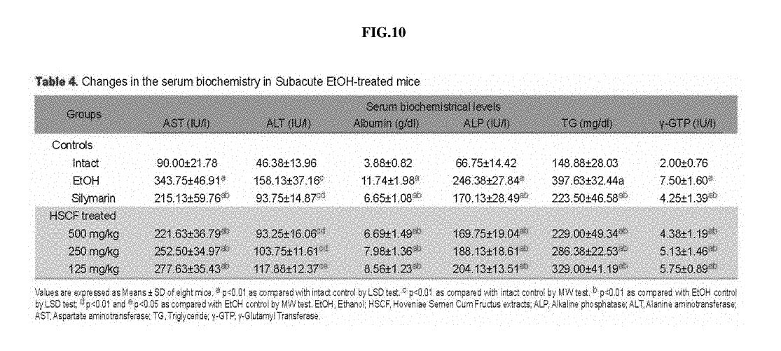

Significant (p<0.01) increases of serum AST, ALT, albumin, ALP, TG and γ-GTP levels were observed in the EtOH control as compared with the intact control. However, the serum chemistries were significantly (p<0.01) decreased by treatment with HSCF extracts at all dosages, and the effect was dose-dependent (Table 4 as shown in FIG. 10).

Changes in the Hepatic TG, TNF-α Contents and CYP 450 2E1 Activity

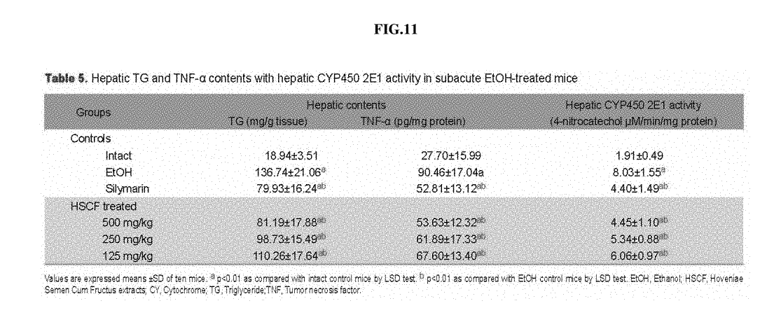

Significant (p<0.01) increases of liver TG contents were observed in the EtOH control as compared with the intact control mice. However, the liver TG contents were significantly (p<0.01) and dose-dependently decreased in HSCF extracts treated mice at all dosages. HSCF extracts at 500 mg/kg showed similar inhibitory effects on the EtOH-induced hepatic TG content elevation as compared with silymarin at 250 mg/kg (Table 5 as shown in FIG. 11).

Significant (p<0.01) increases of liver TNF-α contents were observed in EtOH control as compared with intact control mice. However, the liver TNF-α contents were dose-dependently and significantly (p<0.01) decreased by treatment with HSCF extracts at all dosages. HSCF extracts at 500 mg/kg showed similar inhibitory effects on the EtOH-induced hepatic TNF-α elevation as compared with silymarin at 250 mg/kg in this study (Table 5 as shown in FIG. 11).

Significant (p<0.01) increases of liver CYP450 2E1 activity and hydroxylation of p-nitrophenol to 4-nitrocatechol were observed in the EtOH control as compared with the intact control mice. However, the liver CYP450 2E1 activity was significantly (p<0.01) decreased by treatment with all dosages of HSCF extracts as compared with the EtOH control, dose-dependently. HSCF extracts at 500 mg/kg showed similar inhibitory effects on the EtOH-in-duced hepatic CYP450 2E1 activity increases as compared with silymarin at 250 mg/kg in this experiment (Table 5 as shown in FIG. 11).

Changes in the Hepatic Lipid Peroxidation and Antioxidant Defense Systems

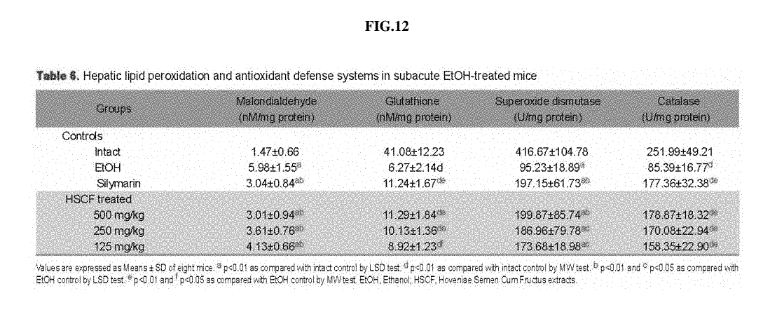

Significant (p<0.01) increases in hepatic lipid peroxidation and increases of MDA contents in liver parenchyma were observed in the EtOH control mice as compared with the intact control mice. However, these increases in liver lipid peroxidation were significantly (p<0.01) and dose-dependently inhibited by treatment with HSCF extracts at 500, 250 and 125 mg/kg as compared with EtOH control mice. HSCF extracts at 500 mg/kg showed similar inhibitory effects on the EtOH-induced hepatic lipid peroxidation as compared with silymarin at 250 mg/kg in our experiment (Table 6 as shown in FIG. 12).

Significant (p<0.01) decreases of hepatic GSH contents, SOD and CAT activities were detected in the EtOH control mice as compared with the intact control. However, hepatic antioxidant defense systems were significantly (p<0.01 or p<0.05) and dose-dependently enhanced by treatment with all dosages of HSCF extracts as compared with the EtOH control, resulting in significantly (p<0.01 or p<0.05) increased hepatic GSH contents, SOD and CAT activities as compared with EtOH control. Similar enhancement effects on the hepatic endogenous antioxidant defense systems were observed in mice treated with HSCF extracts at 500 mg/kg as compared with silymarin at 250 mg/kg (Table 6 as shown in FIG. 12).

Changes in the mRNA Expression of Hepatic Lipogenic Genes

To elucidate the molecular mechanism involved in the aggravation of EtOH-induced steatosis in HSCF extracts treated mice, the expression of genes regulating hepatic lipid synthesis was determined by quantitative RT-PCR, including SREBP-1c, SCD1, ACC1, FAS, PPARγ and DGAT2 in the present study.

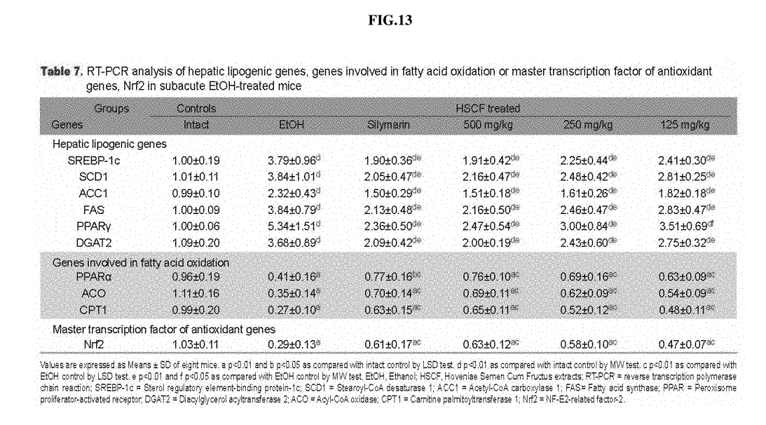

Hepatic SREBP-1c mRNA expression: In the EtOH control mice, significant (p<0.01) increases of hepatic SREBP-1c mRNA expression (SREBP-1c/β-actin mRNA) were observed as compared with the intact control mice. However, significant (p<0.01) dose dependent decreases of the hepatic SREBP-1c mRNA expression were demonstrated in mice treated with HSCF extracts at 500, 250 and 125 mg/kg as compared with the EtOH control mice. HSCF extracts at 500 mg/kg showed similar inhibitory effects on the EtOH-induced increases of hepatic SREBP-1c mRNA expression as compared with silymarin at 250 mg/kg in the present study (Table 7 as shown in FIG. 13).

Hepatic SCD1 mRNA expression: In the EtOH control mice, significant (p<0.01) increases of hepatic SCD1 mRNA expression (SCD1/β-actin mRNA) were observed as compared with the intact control mice. However, significant (p<0.01) and dose-dependent decreases of the hepatic SREBP-1c mRNA expression were observed in mice treated with all three doses of HSCF extracts as compared with the EtOH control mice. Similar inhibitory effects on the hepatic SREBP-1c mRNA expression were demonstrated in mice treated with HSCF extracts at 500 mg/kg as compared with silymarin at 250 mg/kg in this study (Table 7 as shown in FIG. 13).

Hepatic ACC1 mRNA expression: Significant (p<0.01) increases of liver ACC1 mRNA expression (ACC1/β-actin mRNA) were observed in the EtOH control as compared with the intact control mice. However, the hepatic ACC1 mRNA expression was significantly (p<0.01) and dose-dependently decreased by treatment with HSCF extracts at 500, 250 and 125 mg/kg, respectively. HSCF extracts at 500 mg/kg showed similar inhibitory effects on the EtOH-induced hepatic ACC1 mRNA expression increases as compared with silymarin at 250 mg/kg in this experiment (Table 7 as shown in FIG. 13).

Hepatic FAS mRNA expression: In the EtOH control mice, significant (p<0.01) increases of hepatic FAS mRNA expression (FAS/β-actin mRNA) were observed as compared with the intact control mice. However, significant (p<0.01) and dose-dependent decreases of the hepatic FAS mRNA expression were observed with all three doses of HSCF extracts at 500, 250 and 125 mg/kg as compared with the EtOH control mice. HSCF extracts at 500 mg/kg showed similar inhibitory effects on the EtOH-induced increases of hepatic FAS mRNA expression as compared with silymarin at 250 mg/kg (Table 7 as shown in FIG. 13).

Hepatic PPARγ mRNA expression: Significant (p<0.01) increases of liver PPARγ mRNA expression (PPARγ/β-actin mRNA) were observed in the EtOH control as compared with the intact control mice. However, the hepatic PPARγ mRNA expression was significantly (p<0.01 or p<0.05) decreased by treatment with all three doses of HSCF extracts. Similar inhibitory effects on the hepatic PPARγ mRNA expression were observed in mice treated with HSCF extracts at 500 mg/kg as compared with silymarin at 250 mg/kg, in our study (Table 7 as shown in FIG. 13).

Hepatic DGAT2 mRNA expression: In the EtOH control mice, significant (p<0.01) increases of hepatic DGAT2 mRNA expression (DGAT2/β-actin mRNA) were observed as compared with the intact control mice. However, significant (p<0.01) dose dependent decreases of the hepatic DGAT2 mRNA expression were observed in mice treated with HSCF extracts at 500, 250 and 125 mg/kg as compared with EtOH control mice. HSCF extracts at 500 mg/kg showed similar inhibitory effects on the EtOH-induced hepatic DGAT2 mRNA expression increases as compared with silymarin at 250 mg/kg in our experiment (Table 7 as shown in FIG. 13).

Changes in the Hepatic mRNA Expression of Genes Involved in Fatty Acid Oxidation

To elucidate the molecular mechanism involved in the aggravation of EtOH-induced steatosis in HSCF extracts treated mice, the expression of genes involved in fatty acid oxidation was also determined by quantitative RT-PCR, including PPARα, ACO and CPT1 in the present study.

Hepatic PPARα mRNA expression: Significant (p<0.01) decreases of hepatic PPARα mRNA expression (PPARα/β-actin mRNA) were observed in the EtOH control as compared with the intact control mice. However, the hepatic PPARα mRNA expression was significantly (p<0.01) and dose-dependently increased by treatment with all three doses of HSCF extracts at 500, 250 and 125 mg/kg. HSCF extracts at 500 mg/kg showed similar inhibitory effects on the EtOH-induced decreases of hepatic PPARα mRNA expression as compared with silymarin at 250 mg/kg (Table 7 as shown in FIG. 13).

Hepatic ACO mRNA expression: In the EtOH control mice, significant (p<0.01) decreases of hepatic ACO mRNA expression (ACO/β-actin mRNA) were observed as compared with the intact control mice. However, significant (p<0.01) dose dependent increases of the hepatic ACO mRNA expression were observed in mice treated with all three doses of HSCF extracts as compared with the EtOH control mice. Similar up-regulatory effects on the hepatic ACO mRNA expression were observed in mice treated with HSCF extracts at 500 mg/kg as compared to those treated with silymarin at 250 mg/kg (Table 7 as shown in FIG. 13).

Hepatic CPT1 mRNA expression: In the EtOH control mice, significant (p<0.01) decreases of hepatic CPT1 mRNA expression (CPT1/β-actin mRNA) were observed as compared with the intact control mice. However, significant (p<0.01) and dose-dependent increases of the hepatic CPT1mRNA expression were observed in mice treated with HSCF extracts at 500, 250 and 125 mg/kg as compared with the EtOH control mice. HSCF extracts at 500 mg/kg showed similar inhibitory effects on the EtOH-induced hepatic CPT1 mRNA expression decreases as compared with silymarin at 250 mg/kg in our experiment (Table 7 as shown in FIG. 13).

Changes in the Hepatic mRNA Expression of Nrf2

To elucidate the molecular mechanism involved in the aggravation of EtOH-induced oxidative stress in HSCF extracts treated mice, the expression of the master transcription factor of antioxidant gene, Nrf2, was also determined by quantitative RT-PCR in the present study. Significant (p<0.01) decreases of hepatic Nrf2 mRNA expression (Nrf2/β-actin mRNA) were demonstrated in the EtOH control as compared with the intact control mice. However, the hepatic Nrf2 mRNA expression was significantly (p<0.01) and dose-dependently increased by treatment with all three doses of HSCF extracts at 500, 250 and 125 mg/kg. HSCF extracts at 500 mg/kg showed similar inhibitory effects on the EtOH-induced decreases of hepatic Nrf2 mRNA expression as compared with silymarin at 250 mg/kg (Table 7 as shown in FIG. 13).

Effects on the Liver Histopathology

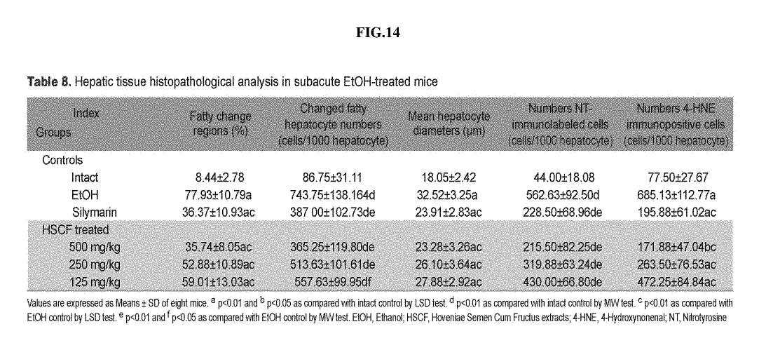

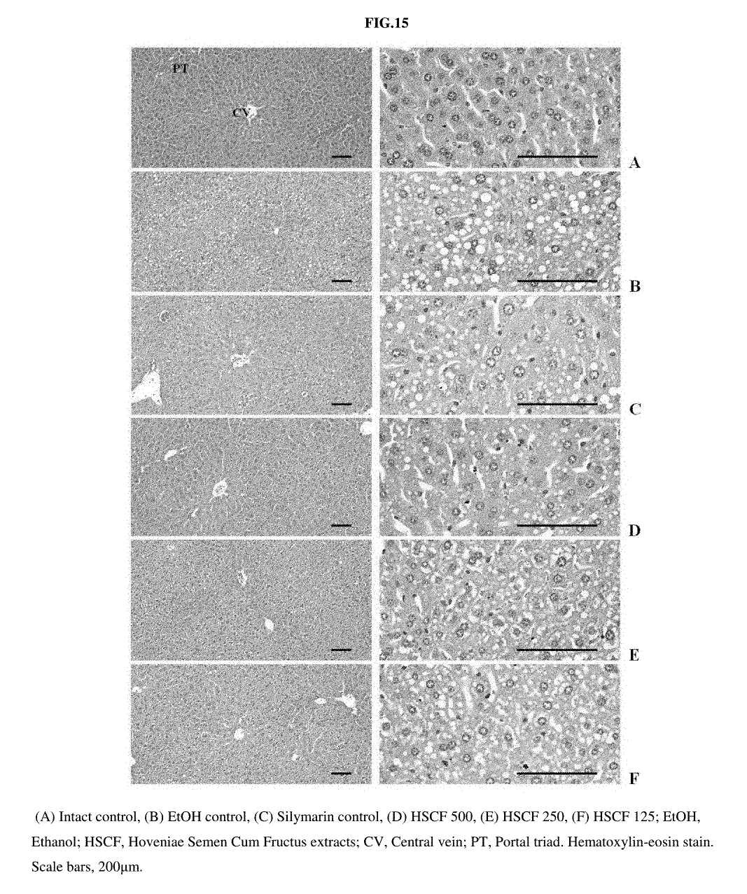

Severe deposition of lipid droplets in the cytoplasm of hepatocytes and hepatosteatosis were observed in all EtOH-dosing groups in the present study. This EtOH-induced hepatosteatosis was re-confirmed with histomorphometry based on the number of changed fatty hepatocytes, mean diameters of hepatocytes and percentage of changed fatty regions, which were significantly (p<0.01) increased in the EtOH control mice as compared with the intact control mice. However, the EtOH treatment-related histopathological hepatosteatosis was significantly (p<0.01 or p<0.05) inhibited by treatment with all three doses of HSCF extracts at 500, 250 and 125 mg/kg as compared with the EtOH control mice, and the effect was dose-dependent. Similar inhibitory effects on the EtOH-induced histopathological hepatosteatosis were observed in mice treated with HSCF extracts at 500 mg/kg as compared to those treated with silymarin at 250 mg/kg in the present study (Table 8 as shown in FIG. 14; FIG. 15).

Effects on the Hepatic NT and 4-HNE-Immunoreactivities

The immunoreactivities of NT as a marker of iNOS related oxidative stress [Pacher et al., 2007] and 4-HNE as a marker of lipid peroxidation [Smathers et al., 2011] in hepatic parenchyma were assessed to determine the liver oxidative stress.

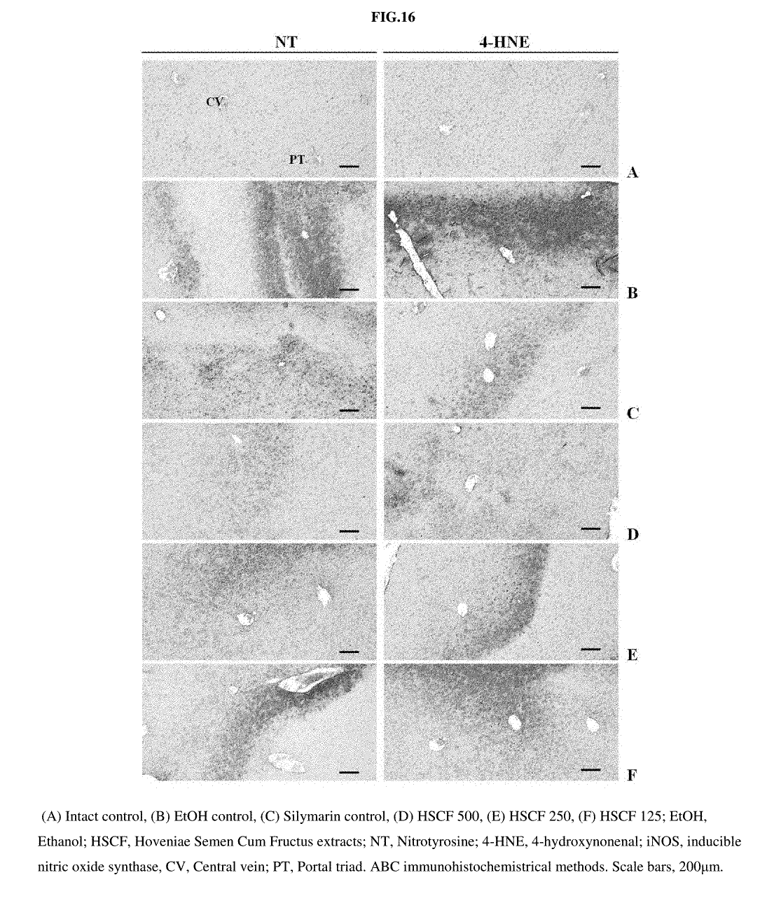

Changes in the NT-immunolabeled hepatocytes: Marked and significant (p<0.01) increases of an iNOS related oxidative stress marker, NT-immunoreactive hepatocytes, were observed in the EtOH control mice as compared with the intact control mice. HSCF extracts at 500, 250 and 125 mg/kg dose-dependently and significantly (p<0.01) normalized the EtOH-related increases of NT-immunoreactive hepatocytes. HSCF extracts at 500 mg/kg showed similar inhibitory effects on the EtOH-induced hepatic NT-immunolabeled cell increases as compared with silymarin at 250 mg/kg in this study (Table 8 as shown in FIG. 13; FIG. 16).

Changes in the 4-HNE-positive hepatocytes: Marked and significant (p<0.01) increases of a lipid peroxidation marker, 4-HNE-immunoreactive hepatocytes, were observed in the EtOH control mice as compared with the intact control mice. However, significant (p<0.01) decreases of the 4-HNE-immunopostive hepatocytes were demonstrated in mice treated with all three doses of HSCF extracts at 500, 250 and 125 mg/kg as compared with the EtOH control mice, and the effect was dose-dependent. HSCF extracts at 500 mg/kg showed similar inhibitory effects on the increases of the hepatic 4-HNE-immunolabeled cells induced by 2 weeks of continuous oral administration of EtOH as compared with silymarin at 250 mg/kg (Table 8 as shown in FIG. 13; FIG. 16).

As cited in the Specification above, the following cited References are hereby incorporated by reference.

Abraham, N. G. & Kappas, A. Pharmacological and clinical aspects of hemeoxygenase. Pharmacol. Rev. 60, 79-127 (2008).

Aebi H. Catalase. In: Bergmeyer HU (Ed.), Methods in Enzymatic Analysis. New York: Academic Press, pp 673-86, 1974.

An, S. W., Kim, Y. G., Kim, M. H. & Lee, B. I. Comparison of hepatic detoxification activity and reducing serum alcohol concentration of Hovenia dulcis Thunb and Alnus japonica Steud. Korean J. Med. Crop. Sci. 7, 263-268 (1999).

Apopa, P. L., He, X. & Ma, Q. Phosphorylation of Nrf2 in the transcription activation domain by casein kinase 2 (CK2) is critical for the nuclear translocation and transcription activation function of Nrf2 in IMR-32 neuroblastoma cells. J. Biochem. Mol. Toxicol. 22, 63-76 (2008).

Bondy, S. C. & Orozco, J. Effects of ethanol treatment upon sources of reactive oxygen species in brain and liver. Alcohol 29, 375-383 (1994).

Bucolo G, David H. Quantitative determination of serum triglycerides by the use of enzymes. Clin Chem. 1973; 19:476-82.

Butler W M, Maling H M, Horning M G, Brodie B B. The direct determination of liver triglycerides. J Lipid Res. 1961; 2:95-6. Bondy S C, Orozco J. Effects of ethanol treatment upon sources of reactive oxygen species in brain and liver. Alcohol Alcohol. 1994; 29:375-83.

Cahill A, Cunningham C C, Adachi M, Ishii H, Bailey S M, Fromenty B, Davies A. Effects of alcohol and oxidative stress on liver pathology: the role of the mitochondrion. Alcohol Clin Exp Res. 2002; 26:907-15.

Castilla R, González R, Fouad D, Fraga E, Muntané J. Dual effect of ethanol on cell death in primary culture of human and rat hepatocytes. Alcohol Alcohol. 2004; 39:290-6.

Cha, P. H. et al. Hovenia dulcis Thunb extract and its ingredient methyl vanillate activate Wnt/β-catenin pathway and increase bone mass in growing or ovariectomized mice. PLoS One 9, e85546 (2014).

Cheeseman K H, Slater T F. An introduction to free radical biochemistry. Br Med Bull. 1993; 49:481-93.

Chen J H, Tipoe G L, Liong E C, So H S, Leung K M, Tom W M, Fung P C, Nanji A A. Green tea polyphenols prevent toxin-induced hepatotoxicity in mice by down-regulating inducible nitric oxide-derived prooxidants. Am J Clin Nutr. 2004; 80:742-51.

Chen Y H, Yang C M, Chang S P, Hu M L. C/EBP beta and C/EBP delta expression is elevated in the early phase of ethanol-induced hepatosteatosis in mice. Acta Pharmacol Sin. 2009; 30:1138-43.

Cooke, M. S., Evans, M. D., Dizdaroglu. M. & Lunec, J. Oxidative DNA damage: mechanisms, mutation, and disease. FASEB J. 17, 1195-1214 (2003).

Crabb D W, Galli A, Fischer M, You M. Molecular mechanisms of alcoholic fatty liver: role of peroxisome proliferator-activated receptor alpha. Alcohol. 2004; 34:35-8.

Dahiru D, Obidoa O. Evaluation of the antioxidant effects of Ziziphus mauritiana Lam. Leaf extracts against chronic ethanol-induced hepatotoxicity in rat liver. Afr J Tradit Complement Altern Med. 2007; 5:39-45.

Das K, Chainy G B. Modulation of rat liver mitochondrial antioxidant defence system by thyroid hormone. Biochim Biophys Acta. 2001; 1537:1-13.

Das S K, Varadhan S, Gupta G, Mukherjee S, Dhanya L, Rao D N, Vasudevan D M. Time-dependent effects of ethanol on blood oxidative stress parameters and cytokines. Indian J Biochem Biophys. 2009; 46:116-21.

DeLeve L D, Kaplowitz N. Glutathione metabolism and its role in hepatotoxicity. Pharmacol Ther. 1991; 52:287-305.

DeLeve, L. D. & Kaplowitz, N. Glutathione metabolism and its role in hepatotoxicity. Pharmacol. Ther. 52, 287-305 (1991).

Devipriya N, Srinivasan M, Sudheer A R, Menon V P. Effect of ellagic acid, a natural polyphenol, on alcohol-induced prooxidant and antioxidant imbalance: a drug dose dependent study. Singapore Med J. 2007; 48:311-8.

Dey A, Cederbaum A I. Alcohol and oxidative liver injury. Hepatology. 2006; 43:563-74.

Diehl A M. Liver disease in alcohol abusers: clinical perspective. Alcohol. 2002; 27:7-11.

Donohue T M Jr. Alcohol-induced steatosis in liver cells. World J Gastroenterol. 2007; 13:4974-8.

Dubinina E E, Dadali V A. Role of 4-hydroxy-trans-2-nonenal in cell functions. Biochemistry (Mosc). 2010; 75:1069-87.

Fischer M, You M, Matsumoto M, Crabb D W. Peroxisome proliferator-activated receptor alpha (PPARalpha) agonist treatment reverses PPARalpha dysfunction and abnormalities in hepatic lipid metabolism in ethanol-fed mice. J Biol Chem. 2003; 278:27997-8004.

Fox J G, Cohen B J, Loew F M. Laboratory animal medicine. Academic Press. Inc., Orlando, 1984.

Franklin, C. C. et al. Structure, function, and post-translational regulation of the catalytic and modifier subunits of glutamate cysteine ligase. Mol. Aspects Med. 30, 86-98 (2009).

Gadelha, A. P. R. et al. Susceptibility of Giardia lamblia to Hovenia dulcis extracts. Parasitol Res. 97, 399-407 (2005).

Galligan J J, Smathers R L, Shearn C T, Fritz K S, Backos D S, Jiang H, Franklin C C, Orlicky D J, Maclean K N, Petersen D R. Oxidative Stress and the ER Stress Response in a Murine Model for Early-Stage Alcoholic Liver Disease. J Toxicol. 2012; 2012:207594.

Ghoneim A I, Eldahshan O A. Anti-apoptotic effects of tamarind leaves against ethanol-induced rat liver injury. J Pharm Pharmacol. 2012; 64:430-8.

Gopumadhavan S, Rafiq M, Azeemuddin M, Mitra S K. Ameliorative effect of Partysmart in rat model of alcoholic liver disease. Indian J Exp Biol. 2008; 46:132-7.

Gouillon Z, Lucas D, Li J, Hagbjork A L, French B A, Fu P, Fang C, Ingelman-Sundberg M, Donohue T M Jr., French S W. Inhibition of ethanol-induced liver disease in the intragastric feeding rat model by chlormethiazole. Proc Soc Exp Biol Med. 2000; 224:302-8.

Guo, J. et al. Myricetin derived from Hovenia dulcis Thunb. ameliorates vascular endothelial dysfunction and liver injury in high choline-fed mice. Food Funct. 6, 1620-1634 (2015).

Hartley D P, Kolaja K L, Reichard J, Petersen D R. 4-Hydroxynonenal and malondialdehyde hepatic protein adducts in rats treated with carbon tetrachloride: immunochemical detection and lobular localization. Toxicol Appl Pharmacol. 1999; 161:23-33.

Hase, K. et al. Hepatoprotective effect of Hovenia dulcis THUNB. on experimental liver injuries induced by carbon tetrachloride or D-galactosamine/lipopolysaccharide. Biol. Pharm. Bull. 20, 381-385 (1997).

Herzig S, Hedrick S, Morantte I, Koo S H, Galimi F, Montminy M. CREB controls hepatic lipid metabolism through nuclear hormone receptor PPAR-gamma. Nature. 2003; 426:190-3.

Ho W Y, Yeap S K, Ho C L, Abdul Rahim R, Alitheen N B. Hepatoprotective activity of Elephantopus scaber on alcohol-induced liver damage in mice. Evid Based Complement Alternat Med. 2012; 2012:417953.

Hu M, Yin H, Mitra M S, Liang X, Ajmo J M, Nadra K, Chrast R, Finck B N, You M. Hepatic-specific lipin-1 deficiency exacerbates experimental alcohol-induced steatohepatitis in mice. Hepatology. 2013; 58:1953-63.

Husain K, Scott B R, Reddy S K, Somani S M. Chronic ethanol and nicotine interaction on rat tissue antioxidant defense system. Alcohol. 2001; 25:89-97.

Hyun, T. K., Eom, S. H., Yu, C. Y. & Roitsch, T. Hovenia dulcis—an Asian traditional herb. Planta Med. 76, 943-949 (2010).

Ishii, T., Itoh, K. & Yamamoto, M. Roles of Nrf2 in activation of antioxidant enzyme genes via antioxidant responsive elements. Methods Enzymol. 348, 182-190 (2002).

Itoh, K., Tong, K. I. & Yamamoto, M. Molecular mechanism activating Nrf2-Keap1 pathway in regulation of adaptive response to electrophiles. Free Radic. Biol. Med. 36, 1208-1213 (2004).

Iwase T. et al. A simple assay for measuring catalase activity: a visual approach. Sci. Rep. 3, 3081 (2013)

Jafri M A, Jalis Subhani M, Javed K, Singh S. Hepatoprotective activity of leaves of Cassia occidentalis against paracetamol and ethyl alcohol intoxication in rats. J Ethnopharmacol. 1999; 66:355-61.

Jamall I S, Smith J C. Effects of cadmium on glutathione peroxidase, superoxidase dismutase and lipid peroxidation in the rat heart: a possible mechanism of cadmium cardiotoxicity. Toxicol Appl Pharmacol. 1985; 80:33-42.

Jenkins R R, Goldfarb A. Introduction: oxidant stress, aging, and exercise. Med Sci Sports Exerc. 1993; 25:210-2.

Jenkins, R. R. & Goldfarb, A. Introduction: oxidant stress, aging, and exercise. Med. Sci. Sports Exerc. 25, 210-212 (1993).

Ji C, Chan C, Kaplowitz N. Predominant role of sterol response element binding proteins (SREBP) lipogenic pathways in hepatic steatosis in the murine intragastric ethanol feeding model. J Hepatol. 2006; 45:717-24.

Ji, Y., Li, J. & Yang, P. Effects of fruits of Hovenia dulcis Thunb on acute alcohol toxicity in mice. Zhong Yao Cai. 24, 126-128 (2001).

Jung Y M, Lee S H, Lee D S, You M J, Chung I K, Cheon W H, Kwon Y S, Lee Y J, Ku S K. Fermented garlic protects diabetic, obese mice when fed a high-fat diet by antioxidant effects. Nutr Res. 2011; 31:387-96.

Kang S J, Lee J E, Lee E K, Jung D H, Song C H, Park S J, Choi S H, Han C H, Ku S K, Lee Y J. Fermentation with Aquilariae Lignum enhances the anti-diabetic activity of green tea in type II diabetic db/db mouse. Nutrients. 2014; 6:3536-71.

Kasdallah-Grissa A, Mornagui B, Aouani E, Hammami M, El May M, Gharbi N, Kamoun A, El-Fazaâ S. Resveratrol, a red wine polyphenol, attenuates ethanol-induced oxidative stress in rat liver. Life Sci. 2007; 80:1033-9.

Kaviarasan S, Anuradha C V. Fenugreek (Trigonella foenum graecum) seed polyphenols protect liver from alcohol toxicity: a role on hepatic detoxification system and apoptosis. Pharmazie. 2007; 62:299-304.

Kavutcu M, Canbolat O, Oztürk S, Olcay E, Ulutepe S, Ekinci C, Gökhun I H, Durak I. Reduced enzymatic antioxidant defense mechanism in kidney tissues from gentamicin-treated guinea pigs: effects of vitamins E and C. Nephron. 1996; 72:269-74.

Kensler, T. W., Wakabayashi, N. & Biswal, S. Cell survival responses to environmental stresses via the Keap1-Nrf2-ARE pathway. Annu. Rev. Pharmacol. Toxicol. 47, 89-116 (2007).

Ki S H, Yang J H, Ku S K, Kim S C, Kim Y W, Cho I J. Red ginseng extract protects against carbon tetrachloride-induced liver fibrosis. J Ginseng Res. 2013; 37:45-53.

Kim H L, Sim J E, Choi H M, Choi I Y, Jeong M Y, Park J, Jung Y, Youn D H, Cho J H, Kim J H, Hwang M W, Jin J S, Hong S H, Cho H W, Um J Y. The AMPK pathway mediates an anti-adipogenic effect of fruits of Hovenia dulcis Thunb. Food Funct. 2014; 5:2961-8.

Kim H S, Park S I, Choi S H, Song C H, Park S J, Shin Y K, Han C H, Lee Y J, Ku S K. Single oral dose toxicity test of blue honeysuckle concentrate in mice. Toxicol Res. 2015; 31:61-8.

Kim, H. L. et al. The AMPK pathway mediates an anti-adipogenic effect of fruits of Hovenia dulcis Thunb. Food Funct. 5, 2961-2968 (2014).

Kobayashi M, Yamamoto M. Molecular mechanisms activating the Nrf2-Keap1 pathway of antioxidant gene regulation. Antioxid. Redox Signal. 2005; 7:385-94.

Kobayashi, M. & Yamamoto, M. Molecular mechanisms activating the Nrf2-Keap1 pathway of antioxidant gene regulation. Antioxid. Redox Signal. 7, 385-394 (2005).

Koch O, Farré S, De Leo M E, Palozza P, Palazzotti B, Borrelo S, Palombini G, Cravero A, Galeotti T. Regulation of manganese superoxide dismutase (MnSOD) in chronic experimental alcoholism: effects of vitamin E-supplemented and -deficient diets. Alcohol Alcohol. 2000; 35:159-63.

Kumar R S, Ponmozhi M, Viswanathan P, Nalini N. Effect of Cassia auriculata leaf extract on lipids in rats with alcoholic liver injury. Asia Pac J Clin Nutr. 2002; 11:157-63.

Kurose I, Higuchi H, Kato S, Miura S, Watanabe N, Kamegaya Y, Tomita K, Takaishi M, Horie Y, Fukuda M, Mizukami K, Ishii H. Oxidative stress on mitochondria and cell membrane of cultured rat hepatocytes and perfused liver exposed to ethanol. Gastroenterology. 199; 112:1331-43.

Kurose, I. et al. Oxidative stress on mitochondria and cell membrane of cultured rat hepatocytes and perfused liver exposed to ethanol. Gastroenterology 112, 1331-1343 (1997).

Lee J M, Li J, Johnson D A, Stein T D, Kraft A D, Calkins M J, Jakel R J, Johnson J A. Nrf2, a multi-organ protector? FASEB J. 2005; 19:1061-6.

Lee, H. Y., Kim, H. S. & Park, Y. S. Hovenodulinol, an active compound extracted from Hovenia dulcis Thunb., a process for preparing the same, and an alcohol decomposing agent or an agent for alleviating lingering intoxication containing the same. Korean patent WO/2002/024678 (2002).

Lee, J. M. et al. Nrf2, a multi-organ protector? FASEB J. 19, 1061-1066 (2005).

Leung T M, Lu Y, Yan W, Morón-Concepción J A, Ward S C, Ge X, Conde de la Rosa L, Nieto N. Argininosuccinate synthase conditions the response to acute and chronic ethanol-induced liver injury in mice. Hepatology. 2012; 55:1596-609.

Levene A. Pathological factors influencing excision of tumours in the head and neck. Part I. Clin Otolaryngol Allied Sci. 1981; 6:145-51.

Levene, A. Pathological factors influencing excision of tumours in the head and neck. Part I. Clin. Otolaryngol. Allied. Sci. 6, 145-151 (1981).

Li G, Min B S, Zheng C, Lee J, Oh S R, Ahn K S, Lee H K. Neuroprotective and free radical scavenging activities of phenolic compounds from Hovenia dulcis. Arch Pharm Res. 2005; 28:804-9.

Li S Y, Yang D, Fu Z J, Woo T, Wong D, Lo A C. Lutein enhances survival and reduces neuronal damage in a mouse model of ischemic stroke. Neurobiol Dis. 2012; 45:624-32.

Li Y M, Chen S H, Yu C H, Zhang Y, Xu G Y. Effect of acute alcoholism on hepatic enzymes and oxidation/antioxidation in rats. Hepatobiliary Pancreat Dis Int. 2004; 3:241-4.

Li, G. et al. Neuroprotective and free radical scavenging activities of phenolic compounds from Hovenia dulcis. Arch. Pharm. Res. 28, 804-809 (2005).

Lieber C. Cytochrome P-4502E1: its physiological and pathological role. Physiol Rev. 1997; 77:517-44.

Lim, S. J., Kim, M., Randy, A. & Nho, C. W. Inhibitory effect of the branches of Hovenia dulcis Thunb. and its constituent pinosylvin on the activities of IgE-mediated mast cells and passive cutaneous anaphylaxis in mice. Food Funct. 6, 1361-1370 (2015).

Lowry O H, Rosenbrough N J, Farr A L, Randall R J: Protein measurement with the Folin phenol reagent. J Biol Chem. 1951; 193:265-75.

Lu, Y., Zhuge, J., Wang, X., Bai, J. & Cederbaum, A. I. Cytochrome P450 2E1 contributes to ethanol-induced fatty liver in mice. Hepatology 47, 1483-1494 (2008).

Ludbrook, J. Update: microcomputer statistics packages. A personal view. Clin. Exp. Pharmacol. Physiol. 24, 294-296 (1997).

McCarty M F. Inhibition of CYP2E1 with natural agents may be a feasible strategy for minimizing the hepatotoxicity of ethanol. Med Hypotheses. 2001; 56:8-11.

Messarah M, Boumendjel A, Chouabia A, Klibet F, Abdennour C, Boulakoud M S, Feki A E. Influence of thyroid dysfunction on liver lipid peroxidation and antioxidant status in experimental rats. Exp Toxicol Pathol. 2010; 62:301-10.

Meyer S A, Kulkarni A P. Hepatotoxicity. In: Hodgson E, Smart RC (ed), Introduction to biochemical toxicology. 3rd ed. New York: John Wiley & Sons, pp 487-90, 2001.

Mohiuddin I, Chai H, Lin P H, Lumsden A B, Yao Q, Chen C. Nitrotyrosine and chlorotyrosine: clinical significance and biological functions in the vascular system. J Surg Res. 2006; 133:143-9.

Moon, S. Y. et al. Tryptanthrin protects hepatocytes against oxidative stress via activation of the extracelluarl signal-regulated kinase/NF-E2-related factor 2 pathway. Biol. Pharm. Bull. 37, 1633-1640 (2014).

Morgan K, French S W, Morgan T R. Production of a cytochrome P450 2E1 transgenic mouse and initial evaluation of alcoholic liver damage. Hepatology. 2002; 36:122-34.

Na, C. S. et al. Anti-fatigue activity of Hovenia dulcis on a swimming mouse model through the inhibition of stress hormone expression and antioxidation. Am. J. Chin. Med. 41, 945-955 (2013).

Na, C. S. et al. Hepatoprotective and blood alcohol lowering effects of fruit peduncle extract of Hovenia dulcis var. Koreana in the in vitro animal models. Yakhak Hoeji 48, 34-40 (2004).

Nagy L E. Molecular aspects of alcohol metabolism: transcription factors involved in early ethanol-induced liver injury. Annu Rev Nutr. 2004; 24:55-78.

Navasumrit P, Ward T H, Dodd N J, O'Connor P J. Ethanol-induced free radicals and hepatic DNA strand breaks are prevented in vivo by antioxidants: effects of acute and chronic ethanol exposure. Carcinogenesis. 2000; 21:93-9.

Noh J R, Kim Y H, Gang G T, Hwang J H, Kim S K, Ryu S Y, Kim Y S, Lee H S, Lee C H. Hepatoprotective effect of Platycodon grandiflorum against chronic ethanolinduced oxidative stress in C57BL/6 mice. Ann Nutr Metab. 2011; 58:224-31.

Nordmann, R. Alcohol and antioxidant systems. Alcohol 29, 513-522 (1994).

Noyan S, Cavusoglu I, Minbay F Z. The effect of vitamin A on EtOH-induced hepatic injuries in rats: a histochemical, immunohistochemical and ultrastructural study. Acta Histochem. 2006; 107:421-34.

Odabasoglu F, Cakir A, Suleyman H, Aslan A, Bayir Y, Halici M, Kazaz C. Gastroprotective and antioxidant effects of usnic acid on indomethacin-induced gastric ulcer in rats. J Ethnopharmacol. 2006; 103:59-65.

Ozaras R, Tahan V, Aydin S, Uzun H, Kaya S, Senturk H. N-acetylcysteine attenuates alcohol-induced oxidative stress in the rat. World J Gastroenterol. 2003; 9:125-8.

Pacher P, Beckman J S, Liaudet L. Nitric oxide and peroxynitrite in health and disease. Physiol Rev. 2007; 87:315-424.

Pari L, Suresh A. Effect of grape (Vitis vinifera L.) leaf extract on alcohol induced oxidative stress in rats. Food Chem Toxicol. 2008; 46:1627-34.

Park J H, Kim S J, Hwang I, Bae K C, Bae J H, Song D K. Green tea extract co-administered with a polymer effectively prevents alcoholic liver damage by prolonged inhibition of alcohol absorption in mice. Alcohol Alcohol. 2013; 48:59-67.

Podder B, Kim Y S, Zerin T, Song H Y. Antioxidant effect of silymarin on paraquat-induced human lung adenocarcinoma A549 cell line. Food Chem Toxicol. 2012; 50:3206-14.

Poli G, Parola M. Oxidative damage and fibrogenesis. Free Radic Biol Med. 1997; 22:287-305.

Ponnappa, B. C. & Rubin E. Modeling alcohol's effects on organs in animal models. Alcohol Res. Health 24, 93-104 (2000).

Rao R K, Seth A, Sheth P. Recent Advances in Alcoholic Liver Disease I. Role of intestinal permeability and endotoxemia in alcoholic liver disease. Am J Physiol Gastrointest Liver Physiol. 2004; 286:G881-4.

Reddy J K, Mannaerts G P. Peroxisomal lipid metabolism. Annu Rev Nutr. 1994; 14:343-70.

Rogers C Q, Ajmo J M, You M. Adiponectin and alcoholic fatty liver disease. IUBMB Life 2008; 60:790-7.

Rouach H, Fataccioli V, Gentil M, French S W, Morimoto M, Nordmann R. Effect of chronic ethanol feeding on lipid peroxidation and protein oxidation in relation to liver pathology. Hepatology. 1997; 25:351-5.

Rouach, H. et al. Effect of chronic ethanol feeding on lipid peroxidation and protein oxidation in relation to liver pathology. Hepatology 25, 351-355 (1997).

Rushworth, S. A., Ogborne, R. M., Charalambos, C. A. & O'Connell, M. A. Role of protein kinase Cdelta in curcumin-induced antioxidant response element-mediated gene expression in human monocytes. Biochem. Biophys. Res. Commun. 341, 1007-1016 (2006).

Saravanan N, Nalini N. Inhibitory effect of Hemidesmus indicus and its active principle 2-hydroxy 4-methoxy benzoic acid on ethanol-induced liver injury. Fundam Clin Pharmacol. 2007; 21:507-14.

Saravanan N, Rajasankar S, Nalini N. Antioxidant effect of 2-hydroxy-4-methoxy benzoic acid on ethanol-induced hepatotoxicity in rats. J Pharm Pharmacol. 2007; 59:445-53.

Saravanan R, Viswanathan P, Pugalendi K V. Protective effect of ursolic acid on ethanol-mediated experimental liver damage in rats. Life Sci. 2006; 78:713-8.

Sedlak J, Lindsay R H. Estimation of total, protein-bound, and nonprotein sulfhydryl groups in tissue with Ellman's reagent. Anal Biochem. 1968; 25:192-205.

Shi S R, Chaiwun B, Young L, Cote R J, Taylor C R. Antigen retrieval technique utilizing citrate buffer or urea solution for immunohistochemical demonstration of androgen receptor in formalin-fixed paraffin sections. J Histochem Cytochem. 1993; 41:1599-604.

Slocum S L, Kensler T W. Nrf2: control of sensitivity to carcinogens. Arch Toxicol. 2011; 85:273-84.

Smathers R L, Galligan J J, Stewart B J, Petersen D R. Overview of lipid peroxidation products and hepatic protein modification in alcoholic liver disease. Chem Biol Interact. 2011; 192107-12.

Sodikoff C H. Laboratory profiles of small animal diseases, A guide to laboratory diagnosis. St. Louise: Mosby; 1995, pp. 1-36.

Somani S M. Exercise, drugs and tissue specific antioxidant system. In: Somani S M (Ed.), Pharmacology in Exercise and Sports. Boca Raton, Fla.: CRC Press, pp 57-95, 1996.

Song, Z. et al. Silymarin protects against acute ethanol-induced hepatotoxicity in mice. Alcohol Clin. Exp. Res. 30, 407-413 (2006).

Steinberg, G. R. & Kemp, B. E. AMPK in health and disease. Physiol. Rev. 89, 1025-1078 (2009).

Subudhi U, Das K, Paital B, Bhanja S, Chainy G B. Alleviation of enhanced oxidative stress and oxygen consumption of L-thyroxine induced hyperthyroid rat liver mitochondria by vitamin E and curcumin. Chem Biol Interact. 2008; 173:105-14.

Sun Y, Larry W O, Ying L. A simple method for clinical assay of superoxide dismutase. Clin Chem. 1988; 34:497-500.

Tajima Y. Biological reference data book on experimental animals. Soft Science Inc., Tokyo, 1989.

Uličná O, Greksák M, Vančová O, Zlatoš L, Galbav\grave{y} Ŝ, Božek P, Nakano M. Hepatoprotective effect of rooibos tea (Aspalathus linearis) on CCl4-induced liver damage in rats. Physiol Res. 2003; 52:461-6.

Venditti P, Di Meo S. Thyroid hormone-induced oxidative stress. Cell Mol Life Sci. 2006; 63:414-34.

Videla L A. Energy metabolism, thyroid calorigenesis, and oxidative stress: functional and cytotoxic consequences. Redox Rep. 2000; 5:265-75.

Voldstedlund M, Tranum-Jensen J, Handberg A, Vinten J. Quantity of Na/K-ATPase and glucose transporters in the plasma membrane of rat adipocytes is reduced by in vivo triiodothyronine. Eur J Endocrinol. 1995; 133:626-34.

Wada S, Yamazaki T, Kawano Y, Miura S, Ezaki O. Fish oil fed prior to ethanol administration prevents acute ethanol-induced fatty liver in mice. J Hepatol. 2008; 49:441-50.

Wang M, Zhu P, Jiang C, Ma L, Zhang Z, Zeng X. Preliminary characterization, antioxidant activity in vitro and hepatoprotective effect on acute alcohol-induced liver injury in mice of polysaccharides from the peduncles of Hovenia dulcis. Food Chem Toxicol. 2012; 50:2964-70.

Wang T, Yang P, Zhan Y, Xia L, Hua Z, Zhang J. Deletion of circadian gene Per1 alleviates acute ethanol-induced hepatotoxicity in mice. Toxicology. 2013; 314:193-201.

Wang, M. et al. Preliminary characterization, antioxidant activity in vitro and hepatoprotective effect on acute alcohol-induced liver injury in mice of polysaccharides from the peduncles of Hovenia dulcis. Food Chem. Toxicol. 50, 2964-2970 (2012).

Wang, T. et al. Deletion of circadian gene Per1 alleviates acute ethanol-induced hepatotoxicity in mice. Toxicology 314, 193-201 (2013).

Wellington K, Jarvis B. Silymarin: a review of its clinical properties in the management of hepatic disorders. BioDrugs. 2001; 15:465-89.

Xiang J, Zhu W, Li Z, Ling S. Effect of juice and fermented vinegar from Hovenia dulcis peduncles on chronically alcohol-induced liver damage in mice. Food Funct. 2012; 3:628-34.

Xing, W. W. et al. Interleukin-22 protects against acute alcohol-induced hepatotoxicity in mice. Biosci. Biotechnol. Biochem. 75, 1290-1294 (2011).

Yang, P. et al. Endogenous A1 adenosine receptor protects mice from acute ethanol-induced hepatotoxicity. Toxicology 309, 100-106 (2013).

Yoshikawa, M. et al. Bioactive constituents of Chinese natural medicines. III. Absolute stereostructures of new dihydroflavonols, hovenitins I, II, and III, isolated from Hoveniae semen seu fructus, the seed and fruit of Hovenia dulcis THUNB. (Rhamnaceae): inhibitory effect on alcohol-induced muscular relaxation and hepatoprotective activity. Yakugaku Zasshi 117, 108-118 (1997).

You M, Crabb D W. Molecular mechanisms of alcoholic fatty liver: role of sterol regulatory element-binding proteins. Alcohol. 2004a; 34:39-43.

You M, Crabb D W. Recent advances in alcoholic liver disease II. Minireview: molecular mechanisms of alcoholic fatty liver. Am J Physiol Gastrointest Liver Physiol. 2004b; 287:G1-6.

Yu S, Matsusue K, Kashireddy P, Cao W Q, Yeldandi V, Yeldandi A V, Rao M S, Gonzalez F J, Reddy J K. Adipocyte-specific gene expressionand adipogenic steatosis in the mouse liver due to peroxisome proliferator-activated receptor gammal (PPARgamma1) overexpression. J Biol Chem. 2003; 278:498-505.

Zarkovic N. 4-hydroxynonenal as a bioactive marker of pathophysiological processes. Mol Aspects Med. 2003; 24:281-91.

Zeng T, Xie K Q. Ethanol and liver: recent advances in the mechanisms of ethanol-induced hepatosteatosis. Arch Toxicol. 2009; 83:1075-81.

Zhang R, Hu Y, Yuan J, Wu D. Effects of Puerariae radix extract on the increasing intestinal permeability in rat with alcohol-induced liver injury. J Ethnopharmacol. 2009; 126:207-14.

Zhang, H. & Forman, H. J. Acrolein induces heme oxygenase-1 through PKC-delta and PI3K in human bronchial epithelial cells. Am. J. Respir. Cell Mol. Biol. 38, 483-490 (2008).

Zhou Z, Sun X, Kang Y. Metallothionein protection against alcoholic liver injury through inhibition of oxidative stress. Exp Biol Med. 2002; 227:214-22.

Zhou Z, Wang L, Song Z, Saari J T, McClain C J, Kang Y J. Zinc supplementation prevents alcoholic liver injury in mice through attenuation of oxidative stress. Am J Pathol. 2005; 166:1681-90.

Zimmermann, K. et al. Activated AMPK boosts the Nrf2/HO-1 signalling axis—a role for the unfolded protein response. Free Radic. Biol. Med. 88, 417-426 (2015).

Having thus described the basic concept of the invention, it will be rather apparent to those skilled in the art that the foregoing detailed disclosure is intended to be presented by way of example only, and is not limiting. Various alterations, improvements, combinations and modifications will occur and are intended to those skilled in the art, though not expressly stated herein. These alterations, improvements, combinations and modifications are intended to be suggested hereby, and are within the spirit and scope of the invention.

1. A pharmaceutical composition for the treatment of liver disease comprising a Hoveniae semen cum fructus extract.

2. The pharmaceutical composition of claim 1, wherein the liver disease is induced by alcohol, drug, stress, or a combination thereof.

3. The pharmaceutical composition of claim 1, wherein NF-E2-related factor-2 (Nrf2) related antioxidant proteins are upregulated.

4. The pharmaceutical composition of claim 1, wherein Nrf2 transactivation is increased.

5. (canceled)

6. (canceled)

7. A dietary supplement, medicinal supplement, or food comprising a Hoveniae semem cum fructus extract.

8. A method for preparing an extract of Hoveniae semem cum fructus comprising:
(i) grinding Hoveniae semem cum fructus to obtain ground Hoveniae semem cum fructus;
(ii) extracting the ground Hoveniae semem cum fructus obtained from step (i) with hot water one to four times at 40-100° C. for 2-10 hours;
(iii) filtering the mixture to obtain a filtrate;
(iv) removing the water from the filtrate under reduced pressure to obtain a residue; and
(v) drying and standardizing the residue using a spray drier 7.4-14.2 ug/g quercetin to obtain an extract of Hoveniae semem cum fructus.

9. The method of claim 8, wherein an excipient selected from the group consisting of dextrin, maltodextrin, and MCC is added for drying the residue.

10. The method of claim 8, wherein
in step (ii) extracting the ground Hoveniae semem cum fructus obtained from step (i) with hot water is performed twice at 95° C. for 4 hours;
and
in step (v) drying and standardizing the residue is performed using the spray drier 11.84 ug/g quercetin to obtain the extract of Hoveniae semem cum fructus.

11. (canceled)

12. A method for treating liver disease comprising the step of administering to a patient the pharmaceutical composition of claim 1, wherein the liver disease is induced by alcohol, drug, stress, or a combination thereof.

13. The method of claim 12, wherein NF-E2-related factor-2 (Nrf2) related antioxidant proteins are upregulated.

14. The method of claim 12, wherein Nrf2 transactivation is increased.

15. (canceled)

16. (canceled)

17. (canceled)

18. A method for protecting against liver disease comprising the step of administering to a patient the supplement of claim 4, wherein the liver disease is induced by alcohol, drug, stress, or a combination thereof.

19. The method of claim 18, wherein NF-E2-related factor-2 (Nrf2) related antioxidant proteins are upregulated.

20. The method of claim 18, wherein Nrf2 transactivation is increased.
